(19) United States
(12) Patent Application Publication
(10) Pub. No.: US 2026/0021147 A1
(43) Pub. Date: Jan. 22, 2026

(54) COMPOSITIONS AND METHODS FOR TREATING INFLAMMATORY DISEASE

(71) Applicant: The Regents of the University of Michigan, Ann Arbor, MI (US)

(72) Inventors: Robert W. P. Glowacki, Ann Arbor, MI (US); Eric C. Martens, Ann Arbor, MI (US)

(21) Appl. No.: 19/114,500

(22) PCT Filed: Sep. 26, 2023
(86) PCT No.: PCT/US2023/075144

§ 371 Date: Mar. 24, 2025

Related U.S. Application Data
(60) Provisional application No. 63/410,282, filed on Sep. 27, 2022.

(51) Int. Cl. A61K 35/74; A61K 45/06; C12N 1/20

(52) U.S. Cl. CPC A61K 35/74; A61K 45/06; C12N 1/20

(57) ABSTRACT

Provided herein are compositions and methods for treating and/or preventing inflammatory disease. In particular, provided herein are non mucin degrading bacteria and their use in treating and/or preventing inflammatory disease (e.g., GvHD).

### STATEMENT OF RELATED APPLICATIONS

This application is a national phase application under 35 U.S.C. § 371 of PCT International Application No.: PCT/US2023/075144, filed on Sep. 26, 2023, which claims priority to and the benefit of U.S. Provisional Patent Application No. 63/410,282, filed Sep. 27, 2022, the entire contents of which are incorporated herein by reference for all purposes.

### STATEMENT REGARDING FEDERALLY SPONSORED RESEARCH OR DEVELOPMENT

This invention was made with government support under DK118024 awarded by the National Institutes of Health. The government has certain rights in the invention.

### SEQUENCE LISTING

The text of the computer readable sequence listing filed herewith, titled “UM-41301-601_SQL.xml”, created Sep. 26, 2023, having a file size of 8,119 bytes, is hereby incorporated by reference in its entirety.

### FIELD OF THE DISCLOSURE

Provided herein are compositions and methods for treating and/or preventing inflammatory disease. In particular, provided herein are non mucin degrading bacteria and their use in treating and/or preventing inflammatory disease (e.g., GvHD and inflammatory bowel diseases).

### BACKGROUND

A variety of disorders are characterized by inflammation. For example, the two major forms of inflammatory bowel disease (IBD), Crohn's Disease and Ulcerative Colitis, are characterized by periods of spontaneous inflammation in the small intestine or colon interspersed with periods of remission. IBD is known to be caused by human genetic predisposition but also the presence of normal commensal gut bacteria.

Graft-versus-host disease (GvHD) is a syndrome, characterized by inflammation in different organs. GvHD is commonly associated with bone marrow transplants and stem cell transplants. White blood cells of the donor's immune system which remain within the donated tissue (the graft) recognize the recipient (the host) as foreign (non-self). The white blood cells present within the transplanted tissue then attack the recipient's body's cells, which leads to GvHD. In the clinical setting, graft-versus-host disease is divided into acute and chronic forms, and scored or graded on the basis of the tissue affected and the severity of the reaction.

In the classical sense, acute graft-versus-host disease is characterized by selective damage to the liver, skin (rash), mucosa, and the gastrointestinal tract. Newer research indicates that other graft-versus-host disease target organs include the immune system (the hematopoietic system, e.g., the bone marrow and the thymus) itself, and the lungs in the form of immune-mediated pneumonitis. Biomarkers can be used to identify specific causes of GvHD, such as elafin in the skin. Chronic graft-versus-host disease also attacks the above organs, but over its long-term course can also cause damage to the connective tissue and exocrine glands.

DNA-based tissue typing allows for more precise HLA matching between donors and transplant patients, which has been proven to reduce the incidence and severity of GvHD and to increase long-term survival. The T-cells of umbilical cord blood (UCB) have an inherent immunological immaturity, and the use of UCB stem cells in unrelated donor transplants has a reduced incidence and severity of GvHD. Methotrexate, cyclosporin and tacrolimus are common drugs used for GvHD prophylaxis.

Intravenously administered glucocorticoids, such as prednisone, are the standard of care in acute GvHD and chronic GvHD. In August 2017 the US FDA approved ibrutinib to treat chronic GvHD after failure of one or more other systemic treatments.

Additional therapies and preventive agents are needed for GvHD and IBD.

### SUMMARY OF THE DISCLOSURE

Provided herein are compositions and methods for treating and/or preventing inflammatory disease. In particular, provided herein are non mucin degrading bacteria and their use in treating and/or preventing inflammatory disease (e.g., GvHD and inflammatory bowel diseases).

The compositions and methods described herein provide a treatment and or preventive agent for inflammatory diseases such as GvHD and IBD. The compositions exhibit low side effects and can be used in combination with existing treatments.

For example, in some embodiments, provided herein is a composition, comprising: a non-mucin degrading Bacteroides thetaiotaomicron comprising a mutation that deletes at least one (e.g., at least 1, 2, 3, 4, 5, 6, 7, 8, 9, 10, or 11) polysaccharide utilization loci (PUL) selected from, for example, BT3172-80, BT1617-36, BT3796-3800, BT3092-3109, BT0752-57, BT2912-23, BT4681-84, BT4634-31, BT0865-67, or BT4250-40. In some exemplary embodiments, PULs BT3172-80, BT1617-36, BT3796-3800, BT3092-3109, BT0752-57, BT2912-23, and BT4681-84; BT3172-80, BT1617-36, BT3796-3800, BT3092-3109, BT0752-57, BT2912-23, BT4681-84, and BT4634-31; BT3172-80, BT1617-36, BT3796-3800, BT3092-3109, BT0752-57, BT2912-23, BT4681-84, BT4634-31, and BT0865-67; or PULS BT3172-80, BT1617-36, BT3796-3800, BT3092-3109, BT0752-57, BT2912-23, BT4681-84, BT4634-31, BT0865-67, and BT4250-40 are deleted. In some cases, the PULS encode one or more enzymes selected from the following glycoside hydrolase (GH) families, for example GH95, sulfatase, GH20, GH2, GH18, GH36, GH29, GH51, GH2, GH88, GH95, GH109, or GH110.

In some embodiments, the composition is a pharmaceutical composition (e.g., comprising a pharmaceutically acceptable carrier), a supplement, or a nutraceutical. In certain embodiments, the composition further comprises one or more polysaccharides selected, for example, arabinan, arabinogalactan, homogalacturonan, rhamnogalacturonan I, pectic galactan, chondroitin sulfate, dextran, α-mannan, or levan.

Additional embodiments provide a composition, comprising: a non-mucin degrading bacteria selected from, for example, Bacteroides caccae Bacteroides fragilis, Bacteroides vulgatus, Bacteroides dorei, Bacteroides fluxus, Bacteroides massiliensis, Bacteroides nordii, Bacteroides ovatus, Parabacteroides merdae, Parabacteroides distasonis, Parabacteroides goldsteinii, or Parabacteroides gordonii wherein the bacteria comprises a mutation that inactivates or eliminates expression of at least one polysaccharide utilization loci (PUL) related to degradation of mucin.

Other embodiments provide the use of a composition described herein to treat or prevent an inflammatory disorder (e.g., including but not limited to, graft vs host disease, inflammatory bowel disease, or Crohn's disease).

Further embodiments provide a method of treating or preventing an inflammatory disease, comprising: administering a composition described herein to a subject in need thereof. In some embodiments, the subject has undergone an organ transplant. In some embodiments, the composition is administered prior to the organ transplant, after the organ transplant, or both. In some embodiments, the composition is administered is administered in multiple doses (e.g., daily, multiple times a day, weekly, monthly, etc.) for a period of time (e.g., days, weeks, months, or years). In some embodiments, the composition is administered orally or fecally (e.g., via fecal transplant). In some embodiments, the composition is in a capsule. In some embodiments, the method further comprises administering an additional treatment for the inflammatory disease (e.g., a corticosteroid). In some embodiments, the non-mucin degrading Bacteroides thetaiotaomicron outcompetes the wild type Bacteroides thetaiotaomicron in the intestine of the subject. In some embodiments, the subject is administered antibiotic prior to the composition.

Additional embodiments provide a method of treating or preventing an inflammatory disease, comprising: administering one or more polysaccharides selected from, for example, arabinan, arabinogalactan, homogalacturonan, rhamnogalacturonan I, pectic galactan, chondroitin sulfate, dextran, α-mannan, or levan to a subject in need thereof, wherein the administering increases the level of mucin in the intestine of the subject.

In certain embodiments, provided herein is a kit or system comprising a composition described herein. In some embodiments, the kit or system further comprises one or more additional components selected from, for example, reagents for assay growth of the bacteria, a polysaccharide, or a test compound.

Also provided is a method of screening a test compound, comprising: a) contacting a composition described herein with a test compound and a polysaccharide; and b) assaying the growth of the bacteria in the presence of the test compound.

Additional embodiments are provided herein.

### DESCRIPTION OF THE FIGURES

FIG. 1 shows that meropenem increased the incidence of intestinal GvHD in allo-HSCT patients and mice. (A) Incidence of intestinal GvHD in 295 allo-HSCT patients. (B) Experimental schema of murine GvHD model. (C) Overall survival. Data are combined from three independent experiments. (D) H&E staining of histological sections of GvHD target organs collected on day 18. Bar, 100 μm. (E) GvHD histology scores of GvHD target organs harvested on day 18. Combined data from two independent experiments are shown as means ±SEM. (F) Experimental schema of murine GvHD model. (G) Bacterial density of fecal samples collected from mice treated as indicated. Data are shown from one representative experiment. (H) Overall survival. Data are combined from three independent experiments.

FIG. 2 shows the effects of meropenem treatment on the composition of the intestinal microbiome in both patients and mice are characterized by expansion of Bacteroides. (A-C) Composition of fecal samples from 44 allo-HSCT patients collected pre-HSCT and on day 14. (A) Bacterial genera composition of fecal samples collected pre-HSCT (top) and on day 14 (bottom). (B-C) Differentially abundant bacterial genera comparing pre-HSCT and day 14 samples collected from (B) meropenem-treated or (C) meropenem-untreated patients, analyzed by the paired-Wilcoxon test and adjusted for false discovery. (D-E) Paired-Wilcoxon test of the genus Bacteroides between at pre-HSCT and on day 14 in (D) meropenem-treated or (E) meropenem-untreated patients. (F) Experimental schema of murine GvHD model. (G) Bacterial density was measured using qPCR of 16S rRNA. (H) Alpha diversity, measured by the inverse Simpson index, was quantified in fecal samples. (I) Principle coordinate analysis (PCoA) of fecal samples. (J) Bacterial genera composition of fecal samples. (K) Differentially 10 abundant bacterial genera comparing fecal samples. (L) Relative abundance of Bacteroides in fecal samples. (G-L) Data are combined from three independent experiments.

FIG. 3 shows that murine BT is associated with meropenem-induced colonic GvHD. (A) Relative abundance of distinguishable Bacteroides sequence variants on day 21. (B) Longitudinal relative abundance of BT. (C) Meropenem-treated mice (FIG. 1C) were stratified by median abundance of BT into a high abundant BT arm (relative abundance>0.2) and low abundant BT arm (relative abundance<0.2). (D) Experimental schema of murine GvHD model using decontamination therapy followed by oral introduction of BT. (E) Overall survival. (A-C, and E) Data are combined from three independent experiments. (F) H&E staining of histological sections of the colon collected on day 28. Bar, 100 μm. (G) GvHD histology scores of the colon collected on day 28.

FIG. 4 shows that meropenem treatment results in increased localization of BT to the colonic mucosa in allogeneic mice. (A) Experimental schema to analyzed paired fecal and mucosal microbiome by 16S rRNA sequencing. (B) PCoA of stool and mucus in distal colon from normal B6D2F1 mice. (C) Ratio of relative abundance of Bacteroidia/Clostridia in stool and mucus from normal B6D2F1 mice. (D) PCoA of stool and mucus in distal colon from allogeneic mice (left) and meropenem-treated allogeneic mice (right). (E) Beta diversity distances within individual mouse. (F) Ratio of relative abundance of Bacteroidia/Clostridia in stool and mucus from allogeneic mice treated or untreated with meropenem. (B-F) Data from one representative experiment are shown.

FIG. 5 shows meropenem-induced compromise of the colonic mucus layer in allogeneic mice. (A) PAS staining of histological colon sections collected on day 18. Bar, 100 μm. The areas inside dotted lines indicate the inner dense colonic mucus layer. (B) Mucus thickness on day 18. Combined data from two independent experiments are shown as means ±SEM. (C) Immunofluorescent staining of colon sections for MUC2 (green) with universal bacterial 16S rRNA gene in situ hybridization probe EUB338 (red) counterstained with DAPI. Bar, 100 μm. Arrowheads indicate infiltrating bacteria. Areas in white squares are magnified and shown below original images. Areas inside dotted lines indicate the inner dense colonic mucus layer. (D) Numbers of bacterial CFUs cultivated from MLNs on day 18. Combined data from three independent experiments are shown. (E) Identified translocated bacteria in MLNs by MALDI 45 Biotyper. Number indicates bacterial CFUs. (F) Immunohistochemistry staining of CD11b in histological colon sections. Bar, 100 μm. (G) Numbers of dendritic cells and neutrophils in the colon were determined by flow cytometric analysis. Combined data from two independent experiments are shown as means ±SEM.

FIG. 6 shows that mucolytic activity of BT is suppressed by ambient xylose (A) Relative expression levels of PULs in BT RNA 5 transcripts sequenced from stool collected from allogeneic mice treated or untreated with meropenem on day 18. Left: PUL identification numbers. Right: enzymatic functional annotations. (B) Relative abundances of monosaccharides of supernatants from stool collected from normal mice and allogeneic mice treated or untreated with meropenem on day 18 measured by IC-MS. (C) Experimental schema of murine GvHD 10 model. (D) PAS staining of histological colon sections collected on day 20. Bar, 100 μm. (E) Mucus thickness on day 20. Data is shown as means ±SEM. (F) Relative abundance of BT on day 20. (G) Correlation analysis of the relative expression levels of PULs in BT RNA transcripts sequenced from stool collected from meropenem-treated allogeneic mice or those with xylose supplementation on day 21. X axis indicates calculated expression levels of PULs in BT RNA transcripts as mean expression levels of meropenem-treated allogeneic mice to allogeneic mice and Y axis indicates calculated expression levels of PULs in BT RNA transcripts as mean expression levels of meropenem-treated allogeneic mice to those with xylose supplementation. (H) Relative expression levels of PULs in BT RNA transcripts. (I) Overall survival. (E-F, I) Data are combined from two independent experiments.

FIG. 7 shows the relative abundances of Clostridia and SCFAs were significantly decreased by meropenem treatment. (A) Meropenem concentrations in the cecal contents of mice were measured 4, 8, 24, 48 and 96 hours after subcutaneously injection of meropenem using LC-MS. (B) Bacterial densities of mouse stool samples collected 7 days after administering with meropenem by drinking water. (C) Relative abundance of the class Clostridia on days 0, 7, 14 and 21. (D) Relative abundances of SCFAs in stool samples from normal mice, allo-HSCT mice and meropenem-treated allo-HSCT mice on day 18 measured by IC-MS.

FIG. 8 shows that levofloxacin and cefepime do not increase the severity of colonic GvHD in allogeneic mice. (A) Experimental schema of murine GvHD model using levofloxacin, cefepime or meropenem treatment. (B) H&E staining of histological sections of the colon collected on day 20 after allo-HSCT. Bar, 100 μm. (C) GvHD histology scores of the colon collected on day 20 after allo-HSCT, quantified by a blinded pathologist. Data are combined from two independent experiments and are shown as means ±SEM. (D) Relative abundances of SCFAs in stool samples from allo-HSCT mice, levofloxacin-, cefepime-, and meropenem-treated allo-HSCT mice on day 20 measured by IC-MS. Data are shown from one representative experiment as means ±SEM.

FIG. 9 shows the intestinal microbiome in allo-HSCT patients treated or untreated with meropenem at pre-HSCT and on day 14. (A) Alpha diversity shown using the inverse Simpson index at pre-HSCT and on day 14. (B) PCoA between meropenem-untreated and treated patients at pre-HSCT (left) and on day 14 (right). (C) Paired-Wilcoxon test of bacteria between at pre-HSCT and on day 14 in meropenem-treated patients (shown in red) or meropenem-untreated patients (shown in blue).

FIG. 10 shows loss of Clostridia and expansion of BT in meropenem-5 treated allogeneic mice and not levofloxacin-or cefepime-treated mice. (A) Stacked bar graphs of bacterial genera composition of fecal samples collected on day 20 after allo-HSCT. (B) Relative abundance of the class Clostridia on day 20. (C) Relative abundance of BT on day 20. (D) PCoA of stool and mucus in distal colon from allogeneic mice (left), allogeneic 10 mice treated with levofloxacin (middle), and allogeneic mice treated with cefepime (right). Numbers shown in each PCoA depict samples collected from the same individual mouse. (E) Beta diversity distances within individual mice. (B-E) Data are shown from one representative experiment.

FIG. 11 shows that mucin-deficient BT did not aggravate the severity of GvHD. (A) Bacterial culture with or without porcine gastric mucin. OD600 nm was measured after 4 hours of anaerobic culture. (B) Levels of porcine gastric mucin in the culture supernatant was determined by using a PAS-based colorimetric assay. (C) Circular plot of open reading frames (ORFs) derived from the complete genome (MDA-JAX BT001). Blue and green bars represent ORFs on the plus strand and the minus strand, respectively. Inner purple-olive ring depicts degree of GC skewing. (D) Bacterial culture of WT BT and mucin-deficient BT in carbohydrate-poor media supplemented with porcine gastric mucin. OD600 nm was measured at 20 hrs. (E) Levels of porcine gastric mucin in the culture supernatant was determined by using a PAS-based colorimetric assay. (F) Experimental schema of murine GvHD model using decontamination therapy followed by oral introduction of WT BT (ATCC 29148) or mucin-deficient BT. (G) Relative abundance of BT in fecal samples collected on day 21. (A-B, D-E, G) Data are shown from one representative experiment as means ±SEM. (F) Overall survival. Data are combined from two independent experiments.

FIG. 12 shows that the mucus-degrading function of BT was not suppressed by glucose in allogeneic mice treated with meropenem. (A) Experimental schema of in vitro bacterial culture assay of BT in media with porcine gastric mucin-containing medium with or without monosaccharides. (B) Relative concentrations of porcine gastric mucin in medium following culture with BT (MDA-JAX BT001). (C) mRNA was extracted from BT pellets cultured with or without monosaccharides and subjected to qPCR analysis of GH2, GH29 and GH33 BT loci. Relative expressions of mRNA were shown by the comparative ΔCt method. (D) PAS staining of histological colon sections collected on day 28. Bar, 100 μm. (E) Mucus thickness on day 28. (F) Bacterial culture with or without the indicated monosaccharide. Monosaccharides were added at 0 hr. (G) Relative expression levels of PULs in BT RNA transcripts. (B-C, E, G) Data are shown from one representative experiment as means ±SEM.

FIG. 13 shows growth of successive deletions of B. theta gene clusters involved in mucin degradation.

FIG. 14 shows successive deletions of B. theta gene clusters involved in mucin degradation.

### DEFINITIONS

As used herein, the term “prokaryotes” refers to a group of organisms that usually lack a cell nucleus or any other membrane-bound organelles. In some embodiments, prokaryotes are bacteria. The term “prokaryote” includes both archaea and eubacteria.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present disclosure.

A “subject” is an animal such as vertebrate, preferably a domestic animal or a mammal. Mammals are understood to include, but are not limited to, murines, simians, humans, bovines, cervids, equines, porcines, canines, felines etc.

An “effective amount” is an amount sufficient to effect beneficial or desired results. An effective amount can be administered in one or more administrations.

“Co-administration” refers to administration of more than one agent or therapy to a subject. Co-administration may be concurrent or, alternatively, the agents or materials described herein may be administered in advance of or following the administration of the other agent(s) or materials. One skilled in the art can readily determine the appropriate dosage for co-administration.

“Administration” refers to the delivery of one or more agents or therapies to a subject. The present disclosure is not limited to a particular mode of administration. In some embodiments, compositions described herein are delivered orally. In some embodiments, compositions are delivered rectally or through another suitable delivery method.

As used herein, the term “toxic” refers to any detrimental or harmful effects on a cell or tissue.

A “pharmaceutical composition” is intended to include the combination of an active agent with a carrier, inert or active, making the composition suitable for diagnostic or therapeutic use in vivo, in vitro, or ex vivo.

As used herein, the term “pharmaceutically acceptable carrier” encompasses any of the standard pharmaceutical carriers, such as a phosphate buffered saline solution, water, and an emulsion, such as an oil/water or water/oil emulsion, and various types of wetting agents. The compositions also can include stabilizers and preservatives. For examples of carriers, stabilizers and adjuvants see Martin, Remington's Pharmaceutical Sciences, 15th Ed., Mack Publ. Co., Easton, Pa. (1975).

As used herein, the term “nutraceutical,” refers to a food substance or part of a food, which includes a bacterium as described herein. Nutraceuticals can provide medical or health benefits, including the prevention, treatment, or cure of a disorder.

As used herein, the terms “probiotic” and “probiotic compositions” are used interchangeable to refer to live microorganisms (e.g., bacteria) that provide health benefits when consumed or otherwise administered (e.g., orally or rectally).

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are gram negative or gram positive. “Gram negative” and “gram positive” refer to staining patterns with the Gram-staining process that is well known in the art. (See e.g., Finegold and Martin, Diagnostic Microbiology, 6th Ed., CV Mosby St. Louis, pp. 13-15 [1982]). “Gram positive bacteria” are bacteria that retain the primary dye used in the Gram stain, causing the stained cells to appear dark blue to purple under the microscope. “Gram negative bacteria” do not retain the primary dye used in the

Gram stain, but are stained by the counterstain. Thus, gram negative bacteria appear red. As used herein, the term “carbohydrate” refers to a biological molecule comprising carbon (C), hydrogen (H) and oxygen (O) atoms, usually with a hydrogen-oxygen atom ratio of 2:1 (e.g., with the empirical formula Cₘ(H₂O)n, m can be different from n. Carbohydrates may be simple (e.g., mono or disaccharides) or complex (e.g., comprising longer straight or branched saccharides). Examples include, but are not limited to, starches, sugars, fibers and the like.

As used herein, the term “taxon” refers to a group of one or more populations of an organism or organisms that form a unit. A taxon is usually known by a particular name and given a particular ranking, especially if and when it is accepted or becomes established. In some embodiments, taxons are defined by NCBI taxonomy systems. In some embodiments, taxons are defined by the sequence of a marker gene (e.g., V4 region of the 16S ribosomal RNA sequence). For example, in some embodiments, bacteria with V4 sequences that are at least 95% identical to the sequences described herein (e.g., at least 95%, 96%, 97%, 98%, 99%, 99.5%, 99.9%, and fractions thereof) are considered as belonging to the same taxon.

### DETAILED DESCRIPTION

Provided herein are compositions and methods for treating and/or preventing inflammatory disease. In particular, provided herein are non mucin degrading bacteria and their use in treating and/or preventing inflammatory disease (e.g., GvHD, inflammatory bowel disease, Crohn's disease, etc.).

The intestinal microbiota is an important modulator of graft-versus-host disease (GvHD), which often complicates allogeneic hematopoietic stem cell transplantation (allo-HSCT). Broad spectrum antibiotics such as carbapenems increase the risk for intestinal GvHD, but mechanisms are not well-understood. Experiments described herein demonstrated that treatment with meropenem, a commonly used carbapenem, aggravates colonic GvHD in mice via expansion of Bacteroides thetaiotaomicron (BT) (ATCC29148 (aka VPI-5482)). BT has a broad ability to degrade dietary polysaccharides and host mucin glycans. BT in meropenem-treated allogeneic mice demonstrated upregulated expression of enzymes involved in degradation of mucin glycans. These mice also had thinning of the colonic mucus layer and decreased levels of xylose in colon luminal contents. Allogeneic mice colonized by mucin-deficient BT showed significantly improved survival compared to those colonized by WT BT.

Accordingly, provided herein are non-mucin degrading bacteria for use in treating and preventing inflammatory diseases such as GvHD.

In some embodiments, the bacteria is a non-mucin degrading Bacteroides thetaiotaomicron that comprises a mutation that deletes at least one polysaccharide utilization loci (PUL) related to mucin metabolism. The present disclosure is not limited to particular PULs. Examples include but are not limited to BT3172-80, BT1617-36, BT3796-3800, BT3092-3109, BT0752-57, BT2912-23, BT4681-84, BT4634-31, BT0865-67, or BT4250-40. In some embodiments, one or more (e.g., 1, 2, 3, 4, 5, 6, 7, 8,9 10, or all 11) of these PULs are deleted. In some exemplary embodiments, PULs BT3172-80, BT1617-36, BT3796-3800, BT3092-3109, BT0752-57, BT2912-23, and BT4681-84; BT3172-80, BT1617-36, BT3796-3800, BT3092-3109, BT0752-57, BT2912-23, BT4681-84, and BT4634-31; BT3172-80, BT1617-36, BT3796-3800, BT3092-3109, BT0752-57, BT2912-23, BT4681-84, BT4634-31, and BT0865-67; or PULs BT3172-80, BT1617-36, BT3796-3800, BT3092-3109, BT0752-57, BT2912-23, BT4681-84, BT4634-31, BT0865-67, and BT4250-40 are deleted.

In some cases, the PULs encode one or more enzymes selected from, for example sulfatases, glycoside hydrolases and M60-like protease (e.g., GH95 (BT3173, BT4682), sulfatase (BT3177, BT1622, BT1624, BT1628, BT1636, BT3796, BT3799, BT3093, BT3095, BT3101, BT3106, BT3107, BT3109, BT0756, BT2914, BT4683), GH20 (BT3178, BT1621, BT1627, BT4681), GH2 (BT3179, BT1626, BT3092, BT0757, BT2922, BT4684), GH18 (BT1632), GH36 (BT3797), GH29 (BT1625, BT3798), GH51 (BT3096), GH88(BT2913), GH109 (BT4243, BT4252), and GH110 (BT4251)). Gene names are in parentheses.

The present disclosure is not limited to non-mucin degrading Bacteroides thetaiotaomicron. In some embodiments, additional mucin degrading bacteria are engineered to delete mucin-degrading genes. Examples include but are not limited to, Bacteroides caccae Bacteroides fragilis, Bacteroides vulgatus, Bacteroides dorei, Bacteroides fluxus, Bacteroides massiliensis, Bacteroides nordii, Bacteroides ovatus, Parabacteroides merdae, Parabacteroides distasonis, Parabacteroides goldsteinii, or Parabacteroides gordonii.

In some embodiments, the non-mucin degrading BT described herein is combined with one or more additional non-mucin degrading bacteria.

In some embodiments, the bacteria are formulated for oral administration. The present disclosure is not limited to particular methods of oral administration. Examples include, but are not limited to, food products, foods, nutraceuticals, nutritional supplements, capsules, etc.

In some embodiments, the bacteria are encapsulated. In some embodiments, a capsule shell that is constructed to dissolve at a predetermined pH of a target region (e.g., large intestine, small intestine, bowel, etc.) is utilized (e.g., available from Assembly Biosciences, Carmel, IN). In some embodiments, capsules also have inner and outer layers that can be engineered to dissolve at different pH levels, making it possible to use a single capsule to deliver two doses of a therapeutic to different locations in the GI tract, or to deliver two different therapeutics to different locations. In some embodiments, mucin is used in the encapsulation technology to protect against stomach acid.

In some embodiments, the bacteria are provided in combination with a carbohydrate (e.g., one or more polysaccharides selected, for example, arabinan, arabinogalactan, homogalacturonan, rhamnogalacturonan I, pectic galactan, chondroitin sulfate, xanthin gum, xanthin gum-derived oligosaccharides, dextran, α-mannan, or levan). In some embodiments, the carbohydrate is encapsulated with the bacteria. In some embodiments, the carbohydrate is provided separately. In some embodiments, the carbohydrate is provided as a food or food product and the bacteria are microencapsulated in or on the food or food product.

In some embodiments, the bacteria are formulated for rectal administration (e.g., as a suppository) or for administration via fecal transplant. Further formulations are described below.

In some embodiments, compositions are formulated as pharmaceutical compositions. The bacteria of embodiments of the disclosure may be administered alone or in combination with pharmaceutically acceptable carriers or diluents, and such administration may be carried out in single or multiple doses.

Compositions may, for example, be in the form of tablets, resolvable tablets, capsules, bolus, drench, pills sachets, vials, hard or soft capsules, aqueous or oily suspensions, aqueous or oily solutions, emulsions, powders, granules, syrups, elixirs, lozenges, reconstitutable powders, liquid preparations, creams, troches, hard candies, sprays, chewing-gums, creams, salves, jellies, gels, pastes, toothpastes, rinses, dental floss and tooth-picks, liquid aerosols, dry powder formulations, HFA aerosols or organic or inorganic acid addition salts.

The pharmaceutical compositions of embodiments of the disclosure may be in a form suitable for oral or rectal administration. Depending upon the disorder and patient to be treated and the route of administration, the compositions may be administered at varying doses.

For oral administration, bacteria of embodiments of the present disclosure may be combined with various excipients. Solid pharmaceutical preparations for oral administration often include binding agents (for example syrups, acacia, gelatin, tragacanth, polyvinylpyrrolidone, sodium lauryl sulphate, pregelatinized maize starch, hydroxypropyl methylcellulose, starches, modified starches, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone and sodium alginate), disintegrants (such as starch and preferably corn, potato or tapioca starch, alginic acid and certain complex silicates, polyvinylpyrrolidone, gelatin, acacia, sodium starch glycollate, microcrystalline cellulose, crosscarmellose sodium, crospovidone, hydroxypropyl methylcellulose and hydroxypropyl cellulose), lubricating agents (such as magnesium stearate, sodium lauryl sulfate, talc, silica polyethylene glycol waxes, stearic acid, palmitic acid, calcium stearate, carnuba wax, hydrogenated vegetable oils, mineral oils, polyethylene glycols and sodium stearyl fumarate) and fillers (including high molecular weight polyethylene glycols, lactose, calcium phosphate, glycine magnesium stearate, starch, rice flour, chalk, gelatin, microcrystalline cellulose, calcium sulphate, and lactitol). Such preparations may also include preservative agents and anti-oxidants.

Liquid compositions for oral administration may be in the form of, for example, emulsions, syrups, or elixirs, or may be presented as a dry product for reconstitution with water or other suitable vehicle before use. Such liquid compositions may contain conventional additives such as suspending agents (e.g. syrup, methyl cellulose, hydrogenated edible fats, gelatin, hydroxyalkylcelluloses, carboxymethylcellulose, aluminium stearate gel, hydrogenated edible fats) emulsifying agents (e.g. lecithin, sorbitan monooleate, or acacia), aqueous or non-aqueous vehicles (including edible oils, e.g. almond oil, fractionated coconut oil) oily esters (for example esters of glycerine, propylene glycol, polyethylene glycol or ethyl alcohol), glycerine, water or normal saline; preservatives (e.g. methyl or propyl p-hydroxybenzoate or sorbic acid) and conventional flavouring, preservative, sweetening or colouring agents. Diluents such as water, ethanol, propylene glycol, glycerin and combinations thereof may also be included.

Other suitable fillers, binders, disintegrants, lubricants and additional excipients are well known to a person skilled in the art. In some embodiments, compositions include one or more of mucin, antioxidants, reductants, or redox-active compound (e.g., to protect bacteria).

In some embodiments, bacteria are spray-dried. In other embodiments, bacteria re-suspended in an oil phase and are encased by at least one protective layer, which is water-soluble (water-soluble derivatives of cellulose or starch, gums or pectins; See e.g., EP 0 180 743, herein incorporated by reference in its entirety).

In some embodiments, compositions are provided as a nutritional or dietary supplement. In some embodiments, the supplement is provided as a powder or liquid suitable for adding by the consumer to a food or beverage. For example, in some embodiments, the dietary supplement can be administered to an individual in the form of a powder, for instance to be used by mixing into a beverage, or by stirring into a semi-solid food such as a pudding, topping, sauce, puree, cooked cereal, or salad dressing, for instance, or by otherwise adding to a food.

The dietary supplement may comprise one or more inert ingredients, especially if it is desirable to limit the number of calories added to the diet by the dietary supplement. For example, the dietary supplement may also contain optional ingredients including, for example, herbs, vitamins, minerals, enhancers, colorants, sweeteners, flavorants, inert ingredients, and the like. For example, the dietary supplement may contain one or more of the following: asorbates (ascorbic acid, mineral ascorbate salts, rose hips, acerola, and the like), dehydroepiandosterone (DHEA), Fo-Ti or Ho Shu Wu (herb common to traditional Asian treatments), Cat's Claw (ancient herbal ingredient), green tea (polyphenols), inositol, kelp, dulse, bioflavinoids, maltodextrin, nettles, niacin, niacinamide, rosemary, selenium, silica (silicon dioxide, silica gel, horsetail, shavegrass, and the like), spirulina, zinc, and the like. Such optional ingredients may be either naturally occurring or concentrated forms.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

In some embodiments, compositions are provided as nutritional supplements (e.g., energy bars or meal replacement bars or beverages). The nutritional supplement may serve as meal or snack replacement and generally provide nutrient calories. In some embodiments, the nutritional supplements provide carbohydrates, proteins, and fats in balanced amounts.

Sources of protein to be incorporated into the nutritional supplement are any suitable protein utilized in nutritional formulations and can include whey protein, whey protein concentrate, whey powder, egg, soy flour, soy milk soy protein, soy protein isolate, caseinate (e.g., sodium caseinate, sodium calcium caseinate, calcium caseinate, potassium caseinate), animal and vegetable protein and mixtures thereof. When choosing a protein source, the biological value of the protein should be considered first, with the highest biological values being found in caseinate, whey, lactalbumin, egg albumin and whole egg proteins. In a preferred embodiment, the protein is a combination of whey protein concentrate and calcium caseinate. These proteins have high biological value; that is, they have a high proportion of the essential amino acids. See Modern Nutrition in Health and Disease, eighth edition, Lea & Febiger, publishers, 1986, especially Volume 1, pages 30-32.

The nutritional supplement can also contain other ingredients, such as one or a combination of other vitamins, minerals, antioxidants, fiber and other dietary supplements (e.g., protein, amino acids, choline, lecithin, omega-3 fatty acids). Selection of one or several of these ingredients is a matter of formulation, design, consumer preference and end-user. Guidance to the amounts or ingredients can be provided by the U.S. RDA doses for children and adults. Further vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D₃; cyanocobalamin; sodium selenite; copper sulfate; vitamin A; vitamin C; inositol; potassium iodide.

Flavors, coloring agents, spices, nuts and the like can be incorporated into the product. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate.

Emulsifiers may be added for stability of the final product. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product.

Preservatives may also be added to the nutritional supplement to extend product shelf life. Preferably, preservatives such as potassium sorbate, sodium sorbate, potassium benzoate, sodium benzoate or calcium disodium EDTA are used.

In some embodiments, the nutritional supplement contains natural or artificial (preferably low calorie) sweeteners, e.g., saccharides, cyclamates, aspartamine, aspartame, acesulfame K, and/or sorbitol. Such artificial sweeteners can be desirable if the nutritional supplement is intended to be consumed by an overweight or obese individual, or an individual with type II diabetes who is prone to hyperglycemia.

The nutritional supplement can be provided in a variety of forms, and by a variety of production methods. In one embodiment, to manufacture a food bar, the liquid ingredients are cooked; the dry ingredients are added with the liquid ingredients in a mixer and mixed until the dough phase is reached; the dough is put into an extruder, and extruded; the extruded dough is cut into appropriate lengths; and the product is cooled. The bars may contain other nutrients and fillers to enhance taste, in addition to the ingredients specifically listed herein.

In some embodiments, compositions are provided as food products, prepared food products, or foodstuffs comprising carbohydrate and bacteria as described above. For example, in some embodiments, beverages and solid or semi-solid foods are provided. These forms can include, but are not limited to, beverages (e.g., soft drinks, milk and other dairy drinks, and diet drinks), baked goods, puddings, dairy products, confections, snack foods, or frozen confections or novelties (e.g., ice cream, milk shakes), prepared frozen meals, candy, snack products (e.g., chips), soups, spreads, sauces, salad dressings, prepared meat products, cheese, yogurt and any other fat or oil containing foods, and food ingredients (e.g., wheat flour).

In some embodiments, the present disclosure provides kits, pharmaceutical compositions, or other delivery systems for use in treating and/or preventing an inflammatory disease in an animal and/or identifying animals in need of treatment with the compositions and methods described herein. The kit may include any and all components necessary, useful or sufficient for research or therapeutic uses including, but not limited to, one or more bacteria, carbohydrate sources, pharmaceutical carriers, PCR primers, reagents, enzymes, buffers, and additional components useful, necessary or sufficient for research, screening, and therapeutic uses. In some embodiments, kits include directions for performing diagnostic assays and/or determining a treatment course of action based on the results of the diagnostic assay. In some embodiments, the kits provide a sub-set of the required components, wherein it is expected that the user will supply the remaining components. In some embodiments, the kits comprise two or more separate containers wherein each container houses a subset of the components to be delivered.

In some embodiments, the kit or system further comprises one or more additional components selected from, for example, reagents for assay growth of the bacteria, a polysaccharide, or a test compound.

Optionally, compositions and kits comprise other active components in order to achieve desired therapeutic effects and/or perform diagnostic assays.

The compositions described herein find use in a variety of research, screening, and therapeutic uses. For example, in some embodiments, the present disclosure provides a method of treating or preventing an inflammatory disease, comprising: administering a composition described herein to a subject in need thereof. In some embodiments, the subject has undergone an organ transplant. In some embodiments, the composition is administered prior to the organ transplant, after the organ transplant, or both. In some embodiments, the composition is administered is administered in multiple doses (e.g., daily, multiple times a day, weekly, monthly, etc.) for a period of time (e.g., days, weeks, months, or years).

In some embodiments, the method further comprises administering an additional treatment for the inflammatory disease (e.g., a corticosteroid). In some embodiments, the non-mucin degrading Bacteroides thetaiotaomicron outcompetes the wild type Bacteroides thetaiotaomicron in the intestine of the subject. In some embodiments, the subject is administered antibiotics prior or at the same time as the composition.

Also provided is a method of screening a test compound, comprising: a) contacting a composition described herein with a test compound and a polysaccharide; and b) assaying the growth of the bacteria in the presence of the test compound.

### EXAMPLES

### Example 1

### Methods

### Data and Code Availability

16S rRNA sequencing data, RNA sequencing data and complete genome data have been deposited at Sequence Read Archive (SRA).

### Retrospective Study Design

295 patients with acute myeloid leukemia (AML) or myelodysplastic syndrome (MDS) underwent allogeneic hematopoietic stem cell transplantation (allo-HSCT) with fludarabine plus busulfan as conditioning therapy and tacrolimus and methotrexate as GvHD prophylaxis from 2011 and 2016 at MD Anderson Cancer Center and were analyzed retrospectively. Patients were classified by antibiotic exposures, including those who received neither cefepime nor meropenem, those who received cefepime alone, those who received meropenem alone and those who received both cefepime and meropenem from day −10 to day after allo-HSCT. Acute GvHD was diagnosed by clinical and/or pathological findings, and graded according to standard criteria (Przepiorka et al., 1995). For patient microbiome analyses, 26 meropenem-unexposed patients and 18 meropenem-exposed patients who underwent allo-HSCT with fludarabine plus busulfan as conditioning therapy from 2014 to 2019 and provided stool samples for the biorepository on day 14 after allo-HSCT were identified.

### Human Samples

Samples were collected from patients undergoing stem cell transplantation and stored at 4° C. for 24-48 hours until aliquoted for long-term storage at −80° C.

### Mice

Female C57BL/6J (B6: H-2b) and B6D2F1 (H-2b/d, CD45.2+) were purchased from The Jackson Laboratory (Bar Harbor, ME). Experiments were performed in a non-blinded fashion.

### Antibiotics Administration

For subcutaneous administration, meropenem was dissolved with PBS and given at a concentration of 10 mg/day. For oral administration, meropenem was dissolved with phosphate buffer pH 8.0 and given at a concentration of 0.625 g/L in the drinking water from day 3 to day 15 after transplant. Levofloxacin and cefepime were supplemented to autoclaved drinking water at a concentration of 0.25 g/L (Schroeder et al., 2001) and 0.625 g/L, respectively, from day 3 to day 15 after transplant. Piperacillin/tazobactam and nystatin were given at a concentration of 3.2 g/L and 320,000 IU/L respectively in combination with meropenem in the drinking water from days 5 to 15 after transplant. In a murine model with a mucin-deficient mutate strain of BT, piperacillin/tazobactam was given at a concentration of 3.2 g/L in combination with vancomycin and metronidazole dosed 1.5 mg daily by oral gavage from days −16 to −12 relative to transplant.

### Xylose and Glucose Administration

D-(+)-xylose (X3877, Sigma-Aldrich) or D-(+)-glucose (G8270, Sigma-Aldrich) was dissolved in phosphate buffer pH 8.0 with meropenem or in water without meropenem and given at a concentration of 0.5% from days 13 to 28 after allo-HSCT.

### HSCT

Mice were transplanted as previously described (Hayase et al., 2017). In brief, after receiving myeloablative total body irradiation (11 Gray) delivered in 2 doses at 4 hour intervals, B6D2F1 (H-2b/d) mice were i.v. injected with 5×10⁶ bone marrow (BM) cells and 5×10⁶ splenocytes from allogeneic B6 (H-2b) or syngeneic B6D2F1 donors. Female mice that were 8 to 12-weeks-old were allocated randomly to each experimental group, ensuring the mean body weight in each group was similar. Total body radiotherapy was performed using a Shepherd Mark I, Model 30, 137Cs irradiator. Mice were maintained in specific pathogen-free (SPF) condition and received normal chow (LabDiet PicoLab Rodent Diet 20 5053, Lab Supply) after HSCT. Survival after HSCT was monitored daily and the degree of clinical GvHD was assessed weekly by using an established scoring system (Cooke et al., 1996).

### Histological and Immunohistochemistry Analysis

For pathological analysis, samples of the small intestine, colon and liver were fixed in 10% formalin, embedded in paraffin, sectioned, and stained with hematoxylin and eosin (H&E). Pathology scores were quantified by a blinded pathologist. For evaluation of mucus thickness, colonic sections containing stool pellets were fixed in methanol-Carnoy's fixative composed of methanol 60%, chloroform 30% and glacial acetic acid 10% and 5 μm sections were made and stained with Periodic acid-Schiff (PAS). Sections were imaged using an Aperio AT2. Mucus thickness of the colonic sections were measured using eSlide Manager Version 12.4.3.5008. Eight measurements per image were taken and averaged over the entire usable colon surface. Immunohistochemistry was performed using primary antibodies of rabbit anti-CD11b (ab75476, Abcam), visualized using 3,3′-diaminobenzidine (DAB) and counterstained using hematoxylin.

### Immunostaining and Fluorescence In Situ Hybridization

Colon containing stool pellets were fixed in methanol-Carnoy's fixative and the 5 μm thin sections were made as described above. Paraffin-embedded sections were dewaxed and hydrated. Sections were incubated with 1 μg Alexa Fluor 594-conjugated EUB338 (5′-GCTGCCTCCCGTAGGAGT-3′) for detection of all bacteria in 200 μL of hybridization buffer (750 mM NaCl, 100 mM Tris-HCl (pH 7.4), 5 mM EDTA, 0.01% BSA, 10% dextran sulfate) at 40° C. for 16 hours (Okumura et al., 2016). Sections were rinsed in wash buffer (50 mM NaCl, 4 mM Tris-HCl (pH 7.4), 0.02 mM EDTA), washed at 45° C. for 20 min, stained with anti-Muc2 antibody [C3] (GTX100664, GeneTex) and counterstained with DAPI (Vector Laboratories). Photographs of sections were obtained using a fluorescent microscope (Nikon NIS Elements, Advanced Research version 4.20).

Sequencing of 16S rRNA Gene Amplicons

Fecal samples which were collected from patients and mice and colonic mucosal samples, which were collected from mice were weighed before DNA isolation. In brief, genomic DNA was isolated using the QIAamp DNA mini kit (51306, Qiagen) according to the manufacturer's protocol that was modified to include an intensive bead-beating lysis step. The V4 region of 16S rRNA gene was amplified by PCR from 100 ng of each of extracted and purified genomic DNA using 515 forward and 806 reverse primer pairs (Caporaso et al., 2012). The quality and quantity of the barcoded amplicons were assessed on an Agilent 4200 TapeStation system (Agilent) and Qubit Fluorometer (Thermo Fisher Scientific), and libraries were prepared after pooling at equimolar ratios. The final libraries were purified using QIAquick gel extraction kit (28706X4, Qiagen) and sequenced with a 2×250 base pair paired-end protocol on the Illumina MiSeq platform.

### Microbiome Data Analysis

Sequencing data from paired-end reads were de-multiplexed using QIIME 2 (Caporaso et al., 2010). Merging of paired-end reads, dereplicating, and length filtering was performed using VSEARCH 2.17.1 (Rognes et al., 2016). Following de-noising and chimera calling using the unoise3 command (Edgar, 2016), unique sequences were taxonomically classified with mothur (Schloss et al., 2009) using the Silva database (Quast et al., 2013) version 138. Weighted UniFrac distances (Lozupone et al., 2011) were determined using QIIME 2, visualized using principal coordinate analysis, and evaluated for statistical significance using permutational multivariate analysis of variance (PERMANOVA) testing. For differential abundance analysis, abundances of sequences belonging to taxonomical groups were included for analysis using the Mann-Whitney U test and adjusted for multiple comparisons using the method of Benjamini-Hochberg. Paired samples were analyzed using the Wilcoxon signed rank test with adjustment for multiple comparisons.

### Quantification of Fecal Bacterial Density

Genomic DNA was isolated from stool as described above. qPCR was performed as previously described (Yang et al., 2015). In brief, 16S rRNA gene sequences were amplified from total fecal DNA using the primers 926F (5′-AAACTCAAAKGAATTGACGG-3′) and 1062R (5′-CTCACRRCACGAGCTGAC-3′). Real-time PCR was carried out in 96-well optical plates on QuantStudio Flex 6 RT PCR (Thermo Fisher) and KAPA SYBR FAST Master Mix (Roche). The PCR conditions included one initial denaturing step of 10 min at 95° C. and 40 cycles of 95° C. for 20 sec and 60° C. for 1 min. Melting-curve analysis was performed after amplification. To determine bacterial density, a plasmid with a 16S rRNA gene of a murine Blautia isolate was generated in the pCR4 backbone and used as a standard.

### Lamina Propria Hematopoietic Cell Dissociation

Murine colons were isolated, dissected longitudinally and then on a shaker in 2% fetal bovine serum in PBS with 1 mM DL-dithiothreitol (Bioworld) at 37° C. for 20 min and subsequently incubated with 1.3 mM EDTA at 37° C. for 40 min. They were rinsed twice and digested with 0.3 mg/ml of type IV collagenase (C5138, Sigma-Aldrich) at 37° C. for 45 min, homogenized, filtered, and washed.

### Flow Cytometric Analysis

Monoclonal antibodies conjugated with fluorescein isothiocynate, phycoerythrin, phycoerythrin-Cy7, peridinin-chlorophyll protein complexes, allophycocyanin, or allophycocyanin-Cy7 were purchased from BioLegend (San Diego, CA,). Lamina propria cells in colon were stained with the antibodies against murine CD45 (30-F11, BioLegend), CD11b (M1/70, BioLegend), CD11c (N418, BioLegend), CD103 (2E7, BioLegend), Ly6G (1A8, BioLegend), MHC-II (M5/114.15.2, BioLegend) and F4/80 (BM8, BioLegend) and Zombie Aqua Fixable viability kit (423101, BioLegend). In flow cytometric analysis, at least 100,000 live samples were analyzed using BD LSRFortessa™ X-20 (BD Biosciences) and FlowJo software (Tree Star, OR). The CD45⁺ cells were classified into neutrophils (CD11b⁺Ly6G⁺) and dendritic cells (CD11c⁺MHC-II⁺CD103⁺).

### Construction of a BT Mucin O-glycan Deficient Mutant

A B. thetaiotaomicron mutant with reduced ability to utilize mucin as a nutrient source was constructed by deleting 11 different PULs that were previously associated with mucin utilization either through direct growth on mucin O-glycans in vitro (Martens et al., 2008) or expression during growth in vivo in the ceca of mice fed a low fiber diet (Bjursell et al., 2006; Sonnenburg et al., 2005). The latter in vivo condition promotes expression of mucin utilization functions. A total of 93 genes encoding 37 annotated enzymes (17 sulfatases, 19 glycoside hydrolases and 1 M60-like protease) were eliminated. PUL deletions were made by allelic exchange using the counter-selectable vector, pExchange-tdk, in a thymidine kinase (tdk) deletion strain to allow counter selection using 5-fluoro-2-deoxy-uridine (FUdR) as previously described (Koropatkin et al., 2008). Briefly, 750 bp flanks were amplified adjacent to each PUL using primers listed in Table 5 and the separate products joined into a single ˜1.5 kbp fragment via overlapping ends. Each fragment was cloned into pExchange-tdk, validated by sequencing and conjugated into either the B. thetaiotaomicron Δtdk parent strain or a previously constructed PUL mutant in order to create the compounded mutant lines after appropriate positive selection using erythromycin and counter-selection using (FUdR) (Koropatkin et al., 2008). The order of gene deletions was as follows, with the numbers being inclusive (i.e., in the first mutant, genes BT3172 and BT3180 were part of the deletion): BT3172-80, BT1617-36 (two tandem PULs removed together), BT3796-3800, BT3092-3109, BT0752-57, BT2912-23, BT4681-84, BT4634-31, BT0865-67, BT4250-40.

### Culturing of Bacteria

Mouse-derived BT (MDA-JAX BT001), Enterococcus faecalis (MDA-JAX EF001) and Clostridium disporicum (MDA-JAX CD001), Clostridium saudiense (MDA-JAX CS001), and Lachnospiraceae unclassified (MDA-JAX LS001) were isolated and cultured from mouse stool samples suspended in 1 ml of chilled 20% anaerobic glycerol in a Whitley anaerobic chamber (10% H₂, 5% CO₂ and 85% N₂). Human-derived BT (ATCC 29148) was purchased from ATCC. Bacterial number was quantified using a Nexcelom Cellometer cell counter with SYTO™ BC dye and propidium iodide. For measuring MICs against meropenem, bacteria were cultured on BYE plates including 5% sterilized rumen fluid (Fisher Scientific) with MIC test strips (Liofilchem™ MTS™ Meropenem [MRP] 0.016-256 μg/mL, Fisher Scientific). Bacterial growth experiments were performed in a bacterial liquid media, BYEM10, composed of a hybrid of BHI and M10 supplemented with yeast extract (Table 4). Bacteria were cultured up to 48 hours at a starting concentration of 1×10⁶ bacteria/ml in BYEM10 broth (pH 7.2) with and without 5 mg/ml of porcine gastric mucin (M1778, Sigma-Aldrich) with or without 0.5 mg/ml of D-(+)-xylose (X3877), D-(+)-mannose (M8574, Sigma-Aldrich) or D-(+)-glucose (G8270). Optical densities (OD600 nm) of bacterial cultures were measured with a BioTek EPOCH 2 plate reader.

### Microbiologic Analysis of Bacterial Translocation

Mesenteric lymph nodes (MLNs) were harvested from mice and homogenized in PBS and cultured anaerobically on BHI plates containing yeast extract and 5% sterilized rumen fluid (Fisher Scientific) and Columbia blood agar plates (BD) for 4 days at 37° C. Colony-forming units (CFUs) were counted and adjusted per organ. Bacteria were identified by MALDI Biotyper.

### Mucin Degradation Assay

Levels of mucin glycans in culture supernatants were determined by a PAS-based colorimetric assay as previously described (Kilcoyne et al., 2011) with minor modifications. Briefly, culture supernatants were centrifuged at 20,000 g, 4° C. for 10 minutes and collected. To perform mucin precipitation, 500 μl of culture supernatants were mixed with 1 mL of molecular grade ethanol and incubated at −30° C. for overnight. Culture supernatants were centrifuged at 20,000 g, 4° C. for 10 minutes. Mucin-containing pellets were washed with 1 mL of molecular grade ethanol twice and resuspended in 500 μl of PBS. 10 μl of washed culture supernatants were transferred into round bottom 96-well plate (Falcon) containing 15 μl of PBS. Serially diluted porcine gastric mucin (Sigma) standards were prepared. Freshly prepared 0.06% periodic acid in 7% acetic acid was added, and incubated at 37° C. for 90 min, followed by 100 μl of Schiff's reagent (84655, Sigma) and incubation at room temperature for 40 min. Absorbance was measured at 550 nm using a BioTek Synergy HTX plate reader.

Short Chain Fatty Acids profiling by ion chromatography-mass spectrometry (IC-MS)

To determine the relative abundance of short chain fatty acids in mouse feces samples, extracts were prepared and analyzed by ultra-high resolution mass spectrometry (HRMS). Fecal pellets were homogenized with a Precellys Tissue Homogenizer. Metabolites were extracted using 1 mL ice-cold 0.1% Ammonium hydroxide in 80/20 (v/v) methanol/water. Extracts were centrifuged at 17,000 g for 5 min at 4° C., and supernatants were transferred to clean tubes, followed by evaporation to dryness under nitrogen. Dried extracts were reconstituted in deionized water, and 5 μL was injected for analysis by IC-MS. IC mobile phase A (MPA; weak) was water, and mobile phase B (MPB; strong) was water containing 100 mM KOH. A Thermo Scientific Dionex ICS-5000+ system included a Thermo IonPac AS11 column (4 μm particle size, 250×2 mm) with column compartment kept at 30° C. The autosampler tray was chilled to 4° C. The mobile phase flow rate was 360 μL/min, and the gradient elution program was: 0-5 min, 1% MPB; 5-25 min, 1-35% MPB; 25-39 min, 35-99% MPB; 39-49 min, 99% MPB; 49-50, 99-1% MPB. The total run time was 50 min. To assist the desolvation for better sensitivity, methanol was delivered by an external pump and combined with the eluent via a low dead volume mixing tee. Data were acquired using a Thermo Orbitrap Fusion Tribrid Mass Spectrometer under ESI negative ionization mode at a resolution of 240,000. Raw data files were imported to Thermo Trace Finder and Compound Discoverer software for spectrum database analysis. The relative abundance of each metabolite was normalized by sample weight.

Pharmacokinetics of meropenem by Triple Quadruple liquid chromatography-mass spectrometry (LC-MS)

C57BL/6 mice were treated with 10 mg of meropenem by subcutaneously injection. Cecal contents were collected prior to, 4, 8, 24, 48, and 96 hours after meropenem injection. To determine the relative abundance of meropenem in mouse cecum samples, extracts were prepared and analyzed with a Thermo Scientific TSQ Quantiva triple quadruple mass spectrometer coupled with a Dionex UltiMate 3000 HPLC system. Approximately 600 mgs of mouse cecal contents were homogenized with a Precellys Tissue Homogenizer. Metabolites were extracted using 100% acetonitrile. The tissue lysates were vortexed, centrifuged at 17,000 g for 5 min 5 at 4° C., and organic layers were transferred to clean tubes, followed by evaporation to dryness under nitrogen. Dried extracts were reconstituted in 50/50 (v/v) water/Acetonitrile, and 5 μL was injected for analysis by LC-MS. The mobile phase A is 100% water and mobile phase B is 0.1% Formic Acid in acetonitrile. Separation of meropenem was achieved on an Agilent SB-C18, 1.8 μm, 100×3 mm column. The flow rate was 250 μL/min at 35° C., and the gradient elution program was: 0-1 min, 5% MPB; 1-5 min, 5-50% MPB; 5-6 min, 50-95% MPB; 6-10 min, 95% MPB; 10-10.1 min, 95-5% MPB. The total run time was 15 min. The mass spectrometer was operated in the MRM positive ion electrospray mode with the transition m/z 384.1->68.0. Raw data files were imported to Thermo Trace Finder software for final analysis. The relative abundance of meropenem was normalized by sample weight.

### Carbohydrates Analysis by IC-MS

To determine the relative abundance of carbohydrates in mouse feces samples, extracts were prepared and analyzed by ultra-HRMS. Fecal pellets were homogenized with a Precellys Tissue Homogenizer. Metabolites were extracted using 1 mL ice-cold 80/20 (v/v) methanol/water. Extracts were centrifuged at 17,000 g for 5 min at 4° C., and supernatants were transferred to clean tubes, followed by evaporation to dryness under nitrogen. Dried extracts were reconstituted in deionized water, and 5 μL was injected for analysis by IC-MS. IC mobile phase A (MPA; weak) was water, and mobile phase B (MPB; strong) was water containing 100 mM KOH. A Thermo Scientific Dionex ICS-5000+ system included a Thermo CarboPac PA-20-Fast column (4 μm particle size, 100×2 mm) with column compartment kept at 30° C. The autosampler tray was chilled to 4° C. The mobile phase flow rate was 200 μL/min, and the gradient elution program was: 0-0.5 min, 1% MPB; 0.5-10 min, 1-5% MPB; 10-15 min, 5-95% MPB; 15-20 min, 95% MPB; 20.5-25, 95-1% MPB. The total run time was 25 min. To assist the desolvation for better sensitivity, methanol was delivered by an external pump and combined with the eluent via a low dead volume mixing tee. Data were acquired using a Thermo Orbitrap Fusion Tribrid Mass Spectrometer under ESI negative ionization mode at a resolution of 240,000. Raw data files were imported to Thermo Trace Finder and Compound Discoverer software for spectrum database analysis. The relative abundance of each metabolite was normalized by sample weight.

### Whole Genome Sequencing of BT (MDA-JAX BT001)

BT (MDA-JAX BT001) genomic DNA was isolated and purified using a Qiagen Genomic-tip 20/G column, according to the manufacturer's instructions. For short-read Illumina sequencing, libraries were constructed with a Nextera DNA Flex Library Prep Kit (Illumina, San Diego, CA, USA), according to the manufacturer's protocol. All libraries were quantified with a TapeStation and pooled in equal molar ratios. The final libraries were sequenced with the NovaSeq 6000 platform (Illumina) to produce 2×150 bp paired-end reads, resulting in ˜5 Gb per sample. For long-read Nanopore sequencing, 500 ng of genomic DNA was used for library preparation using the Rapid Sequencing Kit (SQK-RAD004, Oxford Nanopore Technologies). Libraries were loaded into a FLO-MIN106 flow-cell for 24 h sequencing run on a MinION sequencer platform (Oxford Nanopore Technologies, Oxford, UK). Data acquisition and real-time base calling were carried out by the MinKNOW software version 3.6.5. The fastq files were generated from base called sequencing fast5 reads.

Hybrid assembly and genome annotation of BT (MDA-JAX BT001)

To assemble the complete genome of BT, Flye version 2.8.2. (Kolmogorov et al., 2019) was used with long (Nanopore) reads and short (NovaSeq) reads combined using default settings. The genome was compared to a reference genome (BT VPI-5482) using Rebaler version 0.2.0. (github.com/rrwick/Rebaler) (Wick et al., 2019). The similarities of the genome of MDAJAX BT001 to other reference genomes was calculated using blastn for Bacteroides faecichinchillae (GCF_004801645.1_ASM480164v1), Bacteroides faecis (GCF_000226135.1_ASM22613v2), and BT (VPI-5482), respectively (Altschul et al., 1990). Open reading frames (ORFs) of BT (MDA-JAX BT001) were identified using the Sequence Manipulation Suite (Stothard, 2000) and annotated with polysaccharide utilization loci (PUL) (Terrapon et al., 2018) using BWA version 0.7.17 (Li and Durbin, 2009). The genome of BT and ORFs were depicted using DNA plotter software (Carver et al., 2009).

### RNA Sequencing and Analysis

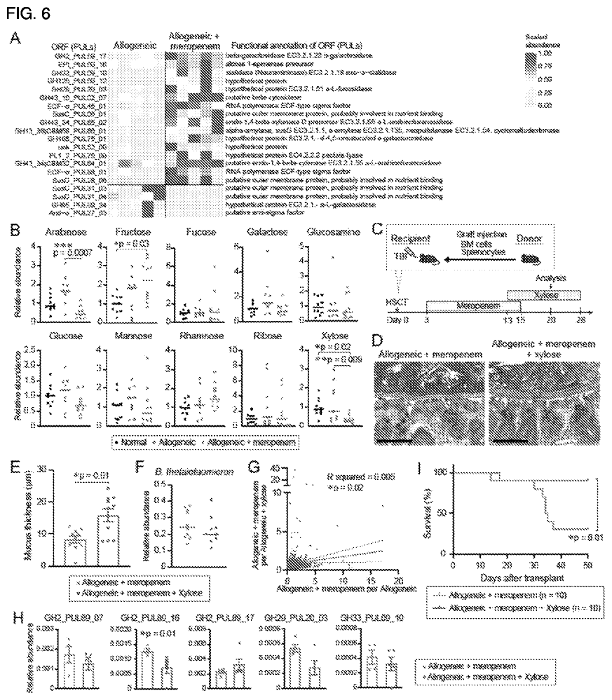

Approximately 30 mg of stool was freshly collected in 700 μL of ice cold Qiazol containing 200 μL of 0.1 mm diameter Zirconia Silica beads (11079101z, BioSpec). Samples were bead beaten twice for 2 min with a 30 second interval recovery. Samples were then centrifuged at 12,000 g for 1 min and the supernatant was collected for RNA isolation using the RNeasy mini kit (74104, Qiagen). RNA was treated on column with DNase I (79254, Qiagen) to eliminate contaminating genomic DNA. RNA quantity and quality was determined using an Agilent 4200 TapeStation system (Agilent). 250 ng of total RNA from mouse stools was used to construct libraries using the Nugen Ovation Complete Prokaryotic RNA-Seq Systems (NuGen) or the Universal Prokaryotic RNA-Seq Library Preparation Kit (9367-32, Tecan) with Unique Dual Indexes (S02480-FG, Tecan), following the manufacturer's protocol. The cDNA libraries were sequenced on the Illumina MiSeq or NovaSeq 6000 system to produce either 1×300 bp single-end reads or 2×150 bp paired-end reads. Sequence data were demultiplexed using QIIME 2 (Caporaso et al., 2010) and their qualities were checked using VSEARCH 2.17.1 (Rognes et al., 2016). Data were filtered and truncated by quality with VSEARCH default settings. The total reads of mouse stool samples were 950923±113406 (mean ±standard deviation) in FIG. 6A and 137623366±38865363 in FIG. 6G-H and 12G. Sequences of ribosomal RNA were removed using BWA software against prokaryotic ribosomal RNA sequences from prokaryotic RefSeq genomes (Tatusova et al., 2016). Sequences of interest were further identified using DIAMOND software version 0.9.24 (Buchfink et al., 2015) to align against PULs. Features with percent identity less than 80% were excluded. The total counts of bacterial isolated samples were 104172±101292 (mean ±standard deviation) in FIG. 6A and 5761717±2518881 in FIG. 6G-H and 12G. Aligned mRNA expression changes were calculated using the Mann-Whitney U test in R software version 3.6.0 via RStudio version 1.2.1335. P values <0.05 were considered statistically significant.

### Quantitative Real-time PCR Analysis of BT Loci

Total RNA was isolated from in vitro cultured BT as described above. The cDNA was synthesized using a High-Capacity cDNA Reverse Transcription Kit (4368814, Thermo Fisher Scientific). The mRNA levels of selected targets were quantified by qPCR using KAPA SYBR FAST Master Mix (Roche) and specific probes (GH2, 5′-CGCACTCTTCTTGCATCTGC-3′ (SEQ ID NO:1) for the forward primer, 5′-TACCAACGGCTCACATTGGG-3′ (SEQ ID NO:2) for the reverse primer; GH29, 5′-GATGCTGGAAAAGGCAACGG-3′ (SEQ ID NO:3) for the forward primer, 5′-AGCGTGCCTTTTCCTTCTGA-3′ (SEQ ID NO:4) for the reverse primer; GH33, 5′-GGTCACCGAAAGACATTATTCATCG-3′ (SEQ ID NO:5) for the forward primer, 5′-GCCGTTTGATACAGATCCATTCC-3′ (SEQ ID NO:6) for the reverse primer) and were normalized to BT specific probes (5′-CACAACAGCCATAGCGTTCCA-3′ (SEQ ID NO:7) for the forward primer, 5′-ATCGCAAAAATAAGATGGGCAAA-3′ (SEQ ID NO:8) for the reverse primer) (Benjdia et al., 2011).

### Statistical Analysis

Data were checked for normality and similar variances between groups and Student's t-tests were used when appropriate. Mann-Whitney U tests were used to compare data between two groups when the data did not follow a normal distribution. The Kaplan-Meier curves were used to depict survival probabilities and the log-rank test was applied to compare survival curves. For clinical data analysis, non-repeated ANOVA was used to compare continuous variables, while chi-square or Fisher's exact tests were used to analyze the frequency distribution between categorical variables. Analyses were performed using R software version 3.6.0 and Prism version 7.0 (GraphPad Software, San Diego, CA). P values <0.05 were considered statistically significant.

### Results

### Meropenem Treatment is Associated With Increased Intestinal GvHD

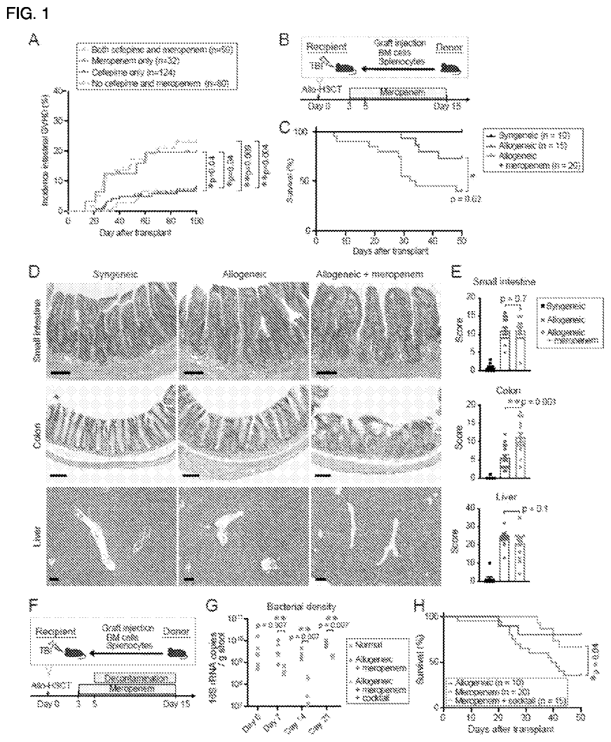

First-line therapy for neutropenic fever is commonly cefepime, an antibiotic that is relatively sparing of commensal anaerobes, while second-line therapy is with meropenem, which is highly active against commensal anaerobes. It was tested whether the use of meropenem was associated with a difference in incidence of intestinal GvHD. 295 patients with acute myeloid leukemia (AML) or myelodysplastic syndrome (MDS) who underwent allo-HSCT with tacrolimus and methotrexate as GvHD prophylaxis following conditioning therapy with fludarabine and busulfan were evaluated for the incidence of acute intestinal GvHD until day 100 after transplant. Patients who received neither cefepime nor meropenem, those who received cefepime alone, those who received meropenem alone, and those who received both cefepime and meropenem during the period from days −10 to 30 relative to allo-HSCT were compared. A summary of clinical characteristics is provided in Table 1; there were no significant differences between each group. The incidence of acute intestinal GvHD was significantly higher in patients who received either meropenem alone or both cefepime and meropenem, while the acute intestinal GvHD incidence in patients receiving only cefepime was similar to that of patients who received neither antibiotic (FIG. 1A). These results indicated that meropenem exposure in allo-HSCT patients is associated with an increased incidence of acute intestinal GvHD.

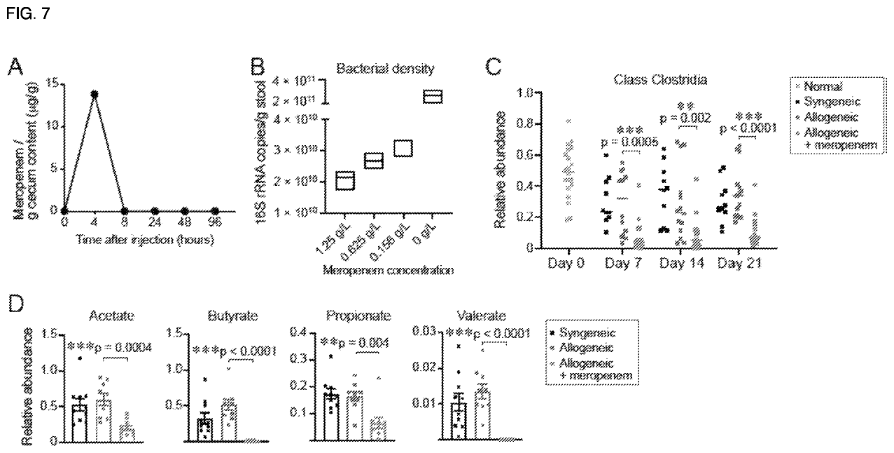

To further explore the relationship between meropenem and intestinal GvHD, the effects of meropenem on GvHD was examined in a mouse model of allo-HSCT. A method of administering meropenem to mice that mimicked the effects of meropenem on the intestinal microbiome of patients was developed. Meropenem concentrations in the cecal lumen of mice were quantified 4, 8, 24, 48 and 96 hours after subcutaneous injection. Subcutaneous injection produced an increase in meropenem concentrations in the cecum at 4 hours after injection, which rapidly declined afterwards (FIG. 7A), indicating that daily or even twice daily dosing in mice was unlikely to produce stable intestinal luminal concentrations of meropenem in mice. Drinking water was then evaluated as a means of continuously administering meropenem to mice, using bacterial density quantified by 16S ribosomal RNA (16S rRNA) gene quantitative PCR (qPCR) to gauge reductions of the intestinal microbiota. A concentration of 0.625 g/L of meropenem, which produced a detectable meropenem concentration in fecal samples (median ±SEM, 0.11±0.07 μg/g stool) and a substantial one-log reduction in fecal bacterial density, was selected as an experimental drinking water dose (FIG. 7B).

Lethally irradiated B6D2F1 (H-2b/d) mice were intravenously injected with 5×10⁶ bone marrow (BM) cells and 5×10⁶ splenocytes from major histocompatibility complex (MHC)-mismatched B6 (H-2b) or syngeneic donors on day 0. Mice at high-risk for GvHD after receiving allogeneic donor-derived cells are referred to as “allogeneic mice,” whereas mice at no risk for GvHD after receiving syngeneic donor-derived cells are referred to as “syngeneic mice.” Meropenem was administered to allogeneic mice in the drinking water from days 3 to 15 relative to allo-HSCT infusion (FIG. 1B). It was found that mice treated with meropenem after allo-HSCT had significantly worsened survival (FIG. 1C), with severe epithelial damage in the colon (FIG. 1D) and significantly higher GvHD histological scores in the colon compared to control allogeneic mice (FIG. 1E). In contrast, GvHD histology in the small intestine and liver were not substantially different in allogeneic mice treated with meropenem (FIG. 1D and 1E). This indicated that meropenem primarily aggravated colonic GvHD.

Studies in the pediatric allo-HSCT population and in mice have found that intestinal Clostridia are associated with protection from GvHD (Simms-Waldrip et al., 2017). Corroborating this, Clostridia are known to be producers of short-chain fatty acids (SCFAs), including butyrate, which plays a role in maintaining epithelial integrity in murine GvHD (Mathewson et al., 2016). It was determined if meropenem-treated allogeneic mice showed a loss of Clostridia abundance and a reduction in SCFA levels. It was found that on day 7, or 4 days following the start of meropenem treatment, Clostridia were indeed depleted and remained so at 6 days after stopping meropenem (FIG. 7C). These mice also had reduced fecal levels of SCFAs, with particularly dramatic reductions in butyrate and valerate (FIG. 7D).

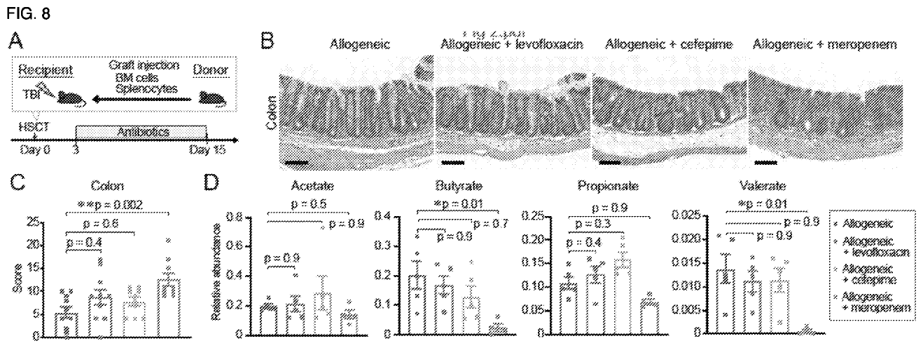

The effects of other antibiotics on colonic GvHD were also evaluated. Levofloxacin, cefepime, or meropenem was administered to allogeneic mice in the drinking water from days 3 to relative to allo-HSCT infusion (FIG. 8A). It was found that allogeneic mice treated with levofloxacin or cefepime experienced colonic GvHD that was similar in severity to control allogeneic mice (FIG. 8B and 8C). Consistent with this, allogeneic mice treated with levofloxacin or cefepime did not show substantially reduced fecal levels of SCFAs, including in particular butyrate and valerate (FIG. 8D).

To determine whether loss of Clostridia during meropenem treatment is mechanistically sufficient to aggravate intestinal GvHD, the effects of intestinal microbiome decontamination was examined in meropenem-treated allogeneic mice. To do so, meropenem and an oral cocktail of piperacillin/tazobactam and nystatin were administered in the drinking water from days 5 to 15 after transplantation (FIG. 1F), similar to a regimen given to pediatric allo-HSCT patients (Bekker et al., 2019). Bacterial density was significantly reduced by this cocktail (FIG. 1G). Intestinal decontamination improved survival in meropenem treated allogeneic mice (FIG. 1H), indicating that meropenem treatment led to worsened GvHD not only due to depletion of beneficial bacteria, but also via expansion of pro-inflammatory bacteria.

### Meropenem Treatment Results in Loss of Clostridia and Expansion of Bacteroides

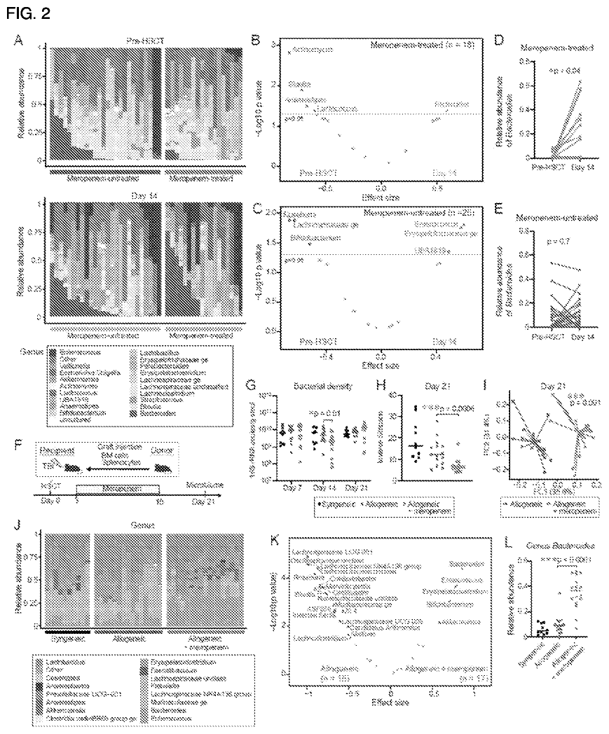

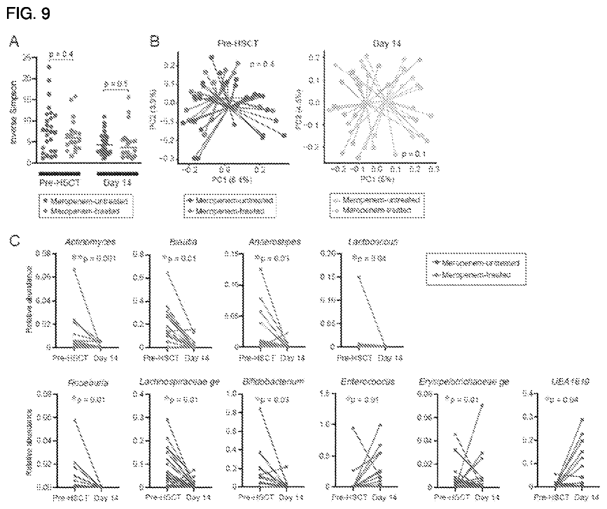

The effects of meropenem on the composition of the colonic microbiota of allo-HSCT patients and mice was examined. Fecal specimens from 44 patients who were treated with allo-HSCT following conditioning with fludarabine and busulfan were collected. Of these patients, 26 did not receive and 18 did receive meropenem treatment between days −10 to 14. Among meropenem-untreated patients, all patients were treated with cefepime, except for one treated with ceftazidime and two patients treated with levofloxacin alone. A comparison of clinical characteristics demonstrated no significant differences between these groups (Table 2). Fecal samples collected at baseline following hospital admission around day-7 as well as on day 14 following allo-HSCT were examined (FIG. 2A). Using PERMANOVA testing of weighted UniFrac beta diversity measures, it was found that at baseline, meropenem untreated and treated patients were not significantly different, nor were they different on day 14 (FIG. 9A and 9B), reflecting a high degree of individual patient intestinal microbiota heterogeneity which has been seen previously in this population and has been attributed to prior antibiotic treatments as well as variable nutrition (Peled et al., 2020). To better characterize microbiome effects of antibiotics in the relatively small and heterogeneous cohort, a paired differential abundance analysis was performed, asking which bacterial genera were the most changed from baseline in the two subgroups of patients. This approach demonstrated that patients treated with meropenem showed significant expansion of bacteria from the genus Bacteroides (FIG. 2A C and 9C). In contrast, patients not treated with meropenem showed expansion of the genus Enterococcus, a genus belonging to Erysipelotrichaceae, and the genus UBA1819, but not Bacteroides (FIG. 2A, 2C, 2E and 9C).

It was next determined if meropenem treatment had similar effects on the composition of the intestinal microbiota of allogeneic mice (FIG. 2F). Using 16S rRNA gene qPCR, fecal bacterial densities were quantified. It was found that meropenem-treated allogeneic mice showed significantly decreased bacterial density on day 14 during meropenem treatment, but by day 21, or 6 days after stopping meropenem, fecal bacterial densities had recovered to a level similar to that of untreated syngeneic and allogeneic mice (FIG. 2G). Most allogeneic mice treated with meropenem began to succumb to aggravated GvHD approximately 3 weeks after allo-HSCT (FIG. 1C). The fecal microbiome was characterized on day 21. It was found that alpha diversity, quantified using the inverse Simpson index, was significantly reduced in meropenem-treated mice (FIG. 2H). PERMANOVA testing demonstrated significant compositional differences between meropenem untreated and treated allogeneic mice (FIG. 2I). Meropenem treatment led to significantly higher abundances of bacteria from several genera, including most substantially Bacteroides, as well as Enterococcus, Erysipelatoclostridium, Bifidobacterium and Akkermansia (FIG. 2J, 2K and 2L). Simultaneously, many genera were depleted, including Blautia, Lachnoclostridium, and other members of Lachnospiraceae, which belong to the class Clostridia (FIG. 2J and 2K).

Bacteroides thetaiotaomicron Contributes to Meropenem-exacerbated Colonic GvHD

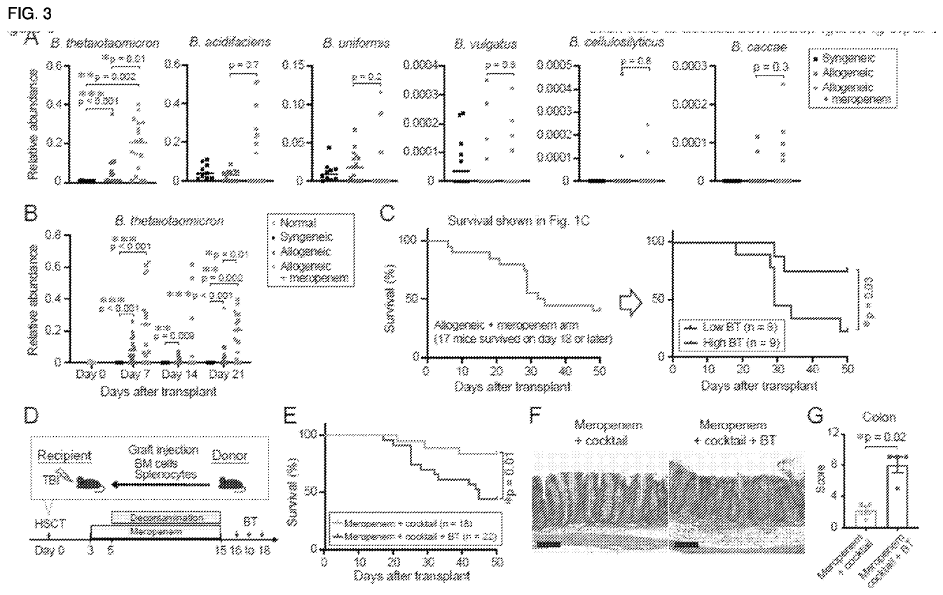

The effects of meropenem treatment on Bacteroides subsets was determined by sequencing the V4 region of the 16S rRNA gene (Jovel et al., 2016). A single Bacteroides sequence variant that was significantly expanded in meropenem-treated mice that had 100% identity with the 16S sequences of BT, Bacteroides faecis, and Bacteroides faecichinchillae while other Bacteroides strains had 98.8% identity or less (FIG. 3A) was identified. The predominant murine Bacteroides isolate was confirmed by whole genome sequencing to be a strain of BT, with 97.4% genomic identity to the ATCC type strain of BT (ATCC 29148), and only 89.2% and 80.8% genomic identity to Bacteroides faecis and Bacteroides faecichinchillae, respectively. This isolate was named MDA-JAX BT001, or murine BT. Murine BT was not eliminated on days 7 and 14 by meropenem-supplemented drinking water and quickly expanded after cessation of meropenem therapy in most meropenem-treated mice, in contrast to Clostridia which consistently remained depleted (FIG. 3B and 7C). These findings indicate that murine BT was less sensitive to meropenem than Clostridia.

This was evaluated by quantifying the minimum inhibitory concentration (MIC) of meropenem against several bacterial isolates (Table 3). Both mouse- and human-derived (ATCC 29148) BT strains showed only moderate sensitivity to meropenem with MICs of 4 μg/mL and 6 μg/mL, respectively. Mouse-derived Enterococcus faecalis (MDA-JAX EF001) was more resistant with an MIC of 12 μg/ml, while MICs of mouse-derived Clostridium disporicum (MDAJAX CD001), Clostridium saudiense (MDA-JAX CS001), and Lachnospiraceae unclassified (MDA-JAX LS001), which belong to the class Clostridia, had MICs of 0.094 μg/mL, 0.38 μg/mL, and 0.38 μg/mL, respectively, showing high sensitivity. Stool collected 48 hours after starting meropenem treatment in normal specific-pathogen-free (SPF) mice had a meropenem concentration of 0.11±0.07 μg/g. These data support that BT can survive during meropenem treatment and then can have a selective advantage after discontinuation of meropenem.

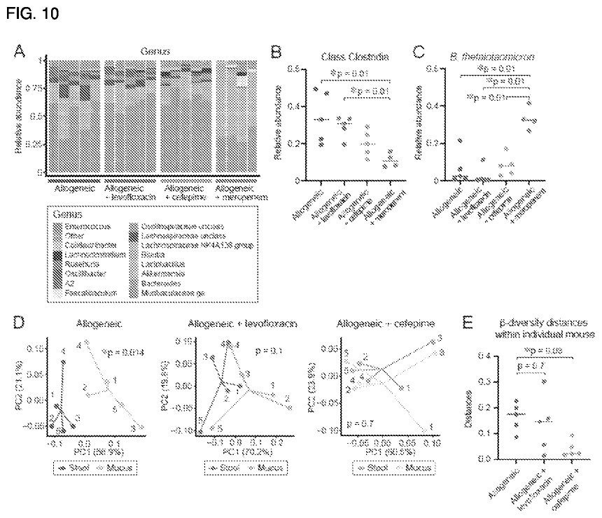

It was determined if loss of Clostridia and increased abundance of BT could be seen following treatment with other antibiotics. Levofloxacin, cefepime or meropenem was administered to mice with GvHD as in FIG. 8A. Levofloxacin-treated mice had significantly higher abundances of Clostridia compared to meropenem-treated mice (FIG. 10A and 10B) and this difference was consistent with fecal butyrate levels (FIG. 8D). Additionally, both levofloxacin- and cefepime treated mice had significantly lower abundances of BT compared to meropenem-treated mice (FIG. 10A and 10C). These results indicated that meropenem is more disruptive of the microbiota, specifically with respect to loss of Clostridia and increased abundance of BT.

To further evaluate for an association between murine BT and aggravated GvHD in meropenem-treated allogeneic mice, mice from 3 experiments shown in FIG. 1C were retrospectively stratified by their median relative abundance of BT. A comparison of these two cohorts showed that mice with higher abundances of BT had worsened overall survival (FIG. 3C). Next, the effects of murine BT on GvHD severity were assayed. In meropenem-treated allogeneic mice that had completed treatment with a decontamination cocktail, 2×10⁷ colony-forming units (CFUs) of murine BT were orally inoculated, followed by monitoring of GvHD severity and survival (FIG. 3D). Allogeneic mice administered murine BT showed worsened survival (FIG. 3E), with severe epithelial damage and significantly higher GvHD histological scores in the colon compared to those without inoculation of murine BT (FIG. 3F and 3G), indicating that murine BT was sufficient to aggravate GvHD in allogeneic mice that had been previously decontaminated.

### Meropenem Treatment Results in Increased Localization of BT to the Intestinal Mucosa

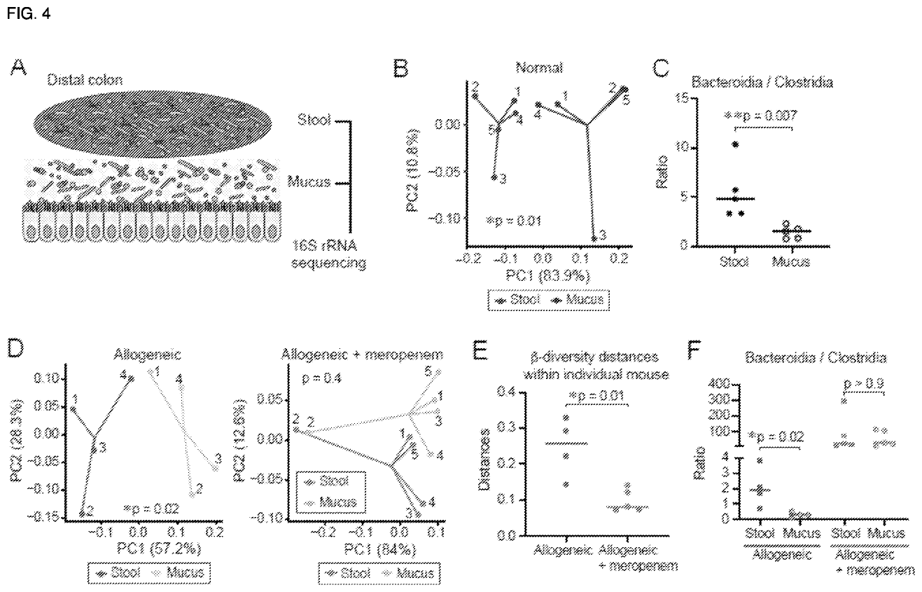

The composition of the intestinal microbiota is known to vary spatially, including longitudinally from proximal to distal, as well as radially from the luminal center to the mucosal surface. The mucosal surface under normal conditions is enriched with bacteria from the phylum Firmicutes including Clostridia, and other intestinal bacteria are relatively excluded, including those belonging to Bacteroidaceae, Enterococcaceae and Lactobacillus, which are enriched in the luminal contents (Nava and Stappenbeck, 2011). It was asked how meropenem treatment can impact the radial composition of the microbiota. The bacterial composition of paired mucosal and fecal pellet samples was evaluated as previously reported (Donaldson et al., 2020) (FIG. 4A). In normal mice, beta diversity analysis identified significant compositional differences between luminal and mucosal samples, and the Bacteroidia-to-Clostridia ratio was significantly higher in luminal samples than mucosal samples (FIG. 4B and 4C). This indicated that Clostridia are enriched in the mucosal surface while Bacteroidia including BT are enriched luminal contents at the steady state. Significant compositional differences between luminal and mucosal samples were found in allogeneic mice (PERMANOVA testing, p=0.02; coefficient in the linear model, R2=0.30), whereas meropenem-treated allogeneic mice showed loss of this distinction (PERMANOVA testing, p=0.4; coefficient in the linear model, R2=0.13) (FIG. 4D). Beta diversity distances between paired luminal and mucosal samples collected from individual mice were reduced in allogeneic mice treated with meropenem, corroborating the other analyses (FIG. 4E). Finally, samples from allogeneic mice showed a significant difference in the Bacteroidia-to-Clostridia ratio when comparing luminal samples with mucosal samples, whereas meropenem-treated allogeneic mice had lost this difference (FIG. 4F). Effects on radial compositional differences with other antibiotics were evaluated. Cefepime-treated allogeneic mice showed loss of compositional differences between luminal and mucosal samples (PERMANOVA testing, p=0.7; coefficient in the linear model, R2=0.05) similar to that seen in meropenem-treated allogeneic mice, whereas levofloxacin treated mice showed less disruption (PERMANOVA testing, p=0.1; coefficient in the linear model, R2=0.17) (FIG. 10D and 10E). Together, these data indicated that in allogeneic mice, the normal enrichment of Clostridia at the mucosal surface was preserved. Meropenem treatment, however, led to disruption of this enrichment, with high relative abundances of Bacteroides seen in mucosal samples. These results are consistent with the hypothesis that Clostridia function to exclude Bacteroidia from the mucosal surface, and that in the setting of reduced Clostridia secondary to eradication by meropenem treatment, Bacteroidia are then able to opportunistically colonize the mucosal surface.

### Meropenem Treatment Induces Thinning of the Colonic Mucus Layer and Impairment of Epithelial Barrier Integrity

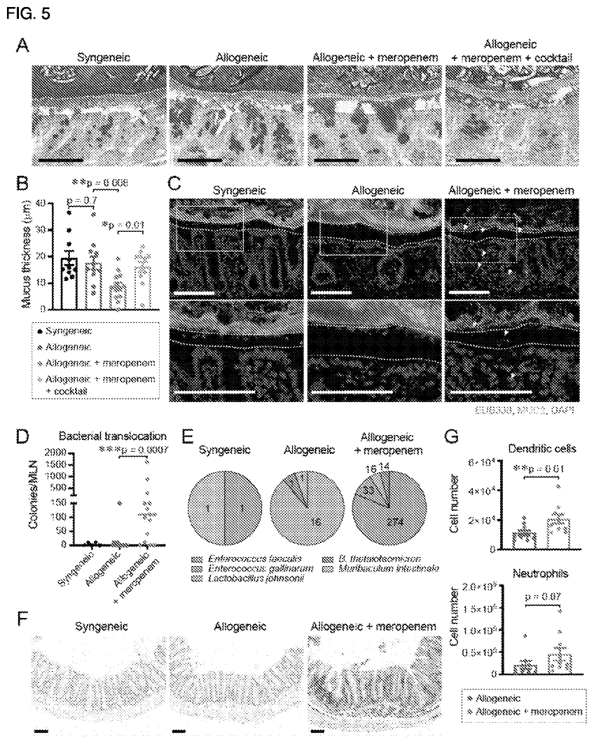

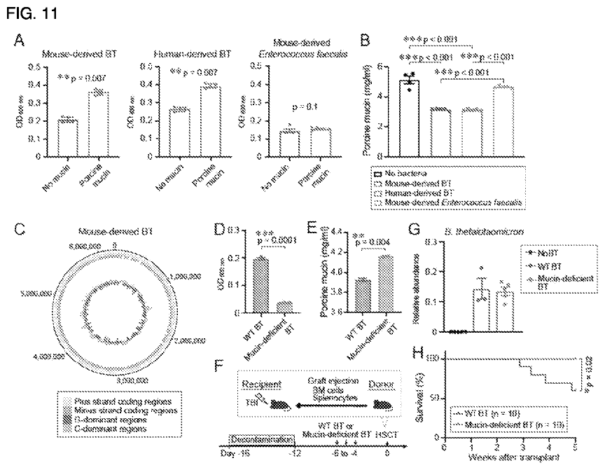

BT is a gram-negative obligate anaerobe with a broad ability to degrade dietary polysaccharides as well as host-derived glycans, including mucins (Bergstrom and Xia, 2013; Tailford et al., 2015). It was hypothesized that meropenem treatment during GvHD could be compromising the colonic mucus layer. The ability of the murine BT isolate (MDA-JAX BT001) to utilize mucin as a carbohydrate source, as well as a human-derived BT strain (ATCC 29148) and, as a comparison, non-mucolytic mouse-derived Enterococcus faecalis (MDA-JAX EF001) was evaluated. It was found that supplementation of carbohydrate-poor media with porcine gastric mucin resulted in augmented growth of both BT strains, in contrast to no growth benefit for murine E. faecalis (FIG. 11A), indicating that mucin-utilization enzymes were expressed by both BT strains. Degradation of mucin-derived carbohydrates was quantified using a PASbased colorimetric assay and it was confirmed that both BT strains displayed degradation of mucin derived carbohydrates (FIG. 11B). It was then asked if meropenem treatment impacted the dense colonic mucus layer of allogeneic mice. PAS staining of histological sections demonstrated a significantly thinned colonic mucus layer in meropenem-treated allogeneic mice compared to untreated allogeneic mice and syngeneic mice (FIG. 5A and 5B). Decontamination, in contrast, led to preservation of this mucus layer in meropenem-treated allogeneic mice, indicating that meropenem was leading to increased mucus degradation by colonic bacteria (FIG. 5A and 5B). The mucus layer provides a critical contribution to the intestinal barrier by excluding bacteria from the intestinal lumen adjacent to the intestinal epithelium. Histological sections were stained with 16S fluorescence in situ hybridization (FISH) probes and dissemination of bacteria into the mucus layer and lamina propria of the colon was visualized in meropenem-treated allogeneic mice (FIG. 5C). To better quantify effects of meropenem on bacterial translocation, MLNs were cultivated microbiologically. Higher bacterial loads were found in meropenem-treated allogeneic mice. Translocating bacteria included BT (FIG. 5D and 5E). Bacterial translocation has previously been found to aggravate GvHD via at least two mechanisms, including recruitment of neutrophils, which can compound tissue damage, as well as by enhancing antigen presentation by dendritic cells through activation of pathogen-associated molecular pattern signaling pathways (Koyama et al., 2015; Schwab et al., 2014). It was thus determined if this barrier compromise led to an inflammatory response in meropenem-treated mice and indeed observed marked colonic tissue infiltration by neutrophils and dendritic cells (FIG. 5F and 5G). Altogether, these findings indicated that meropenem treatment led to compromise of the colonic mucus layer, increased bacterial translocation and an aggravated inflammatory response.

### Meropenem Treatment Upregulates In Vivo Expression of Mucus-degrading Enzymes by BT

While meropenem treatment led to higher abundances of BT in allogeneic mice, GvHD itself, without meropenem treatment, also resulted in moderate increases in abundances of BT compared to syngeneic mice (FIG. 3B). Allogeneic mice, however, did not display a notable thinning of the mucus layer or barrier compromise, despite increased intestinal abundances of BT. This led us to a determination if meropenem treatment during GvHD may lead to alterations in the behavior of BT. To evaluate this further, RNA sequencing of stool samples from allo-HSCT mice without and with meropenem treatment was performed. The genome of the mouse-derived BT isolate (MDA-JAX BT001) (FIG. 11C) evaluated. Open-reading frames (ORF) were assigned to polysaccharide utilization loci (PUL) using the Polysaccharide-Utilization Loci DataBase (PULDB) (Terrapon et al., 2018) (Data S1). RNA reads that aligned to the BT genome were examined. It was found that meropenem treatment led to upregulated expression in murine BT of GH2 β-galactosidase, GH33 sialidase and GH29 α-Lfucosidase, all of which likely participate in the degradation of host mucin glycans (FIG. 6A).

To characterize the contribution of its mucus-degrading capabilities when BT exacerbates GvHD, the effects of a mucin-deficient BT strain was evaluated in a murine GvHD model. This BT strain was engineered to be deficient in 11 PULs that contribute to mucin glycan degradation. This strain grew poorly in culture media containing porcine gastric mucin as the primary carbohydrate source and showed significantly decreased degradation of mucin-derived carbohydrates quantified by a PAS-based colorimetric assay compared to wild-type (WT, ATCC 29148) BT (FIG. 11D and 11E). A 5-day antibiotic regimen composed of piperacillin/tazobactam was administered in the drinking water and oral gavage of both vancomycin and metronidazole to eliminate endogenous Bacteroides from the intestinal microbiome in recipient mice. Bacteroides free mice were inoculated with either WT or mucin-deficient BT orally once daily for 3 days, followed 4 days later by allo-HSCT (FIG. 11F). On day 21 after allo-HSCT, BT was abundant in fecal pellets from recipients that received WT BT or mucin-deficient BT but was absent in control mice (FIG. 11G). Allogeneic mice colonized by mucin-deficient BT showed significantly improved survival compared to those colonized by WT BT (FIG. 11H), supporting that mucus-degrading capabilities of BT contribute to exacerbation of GvHD.

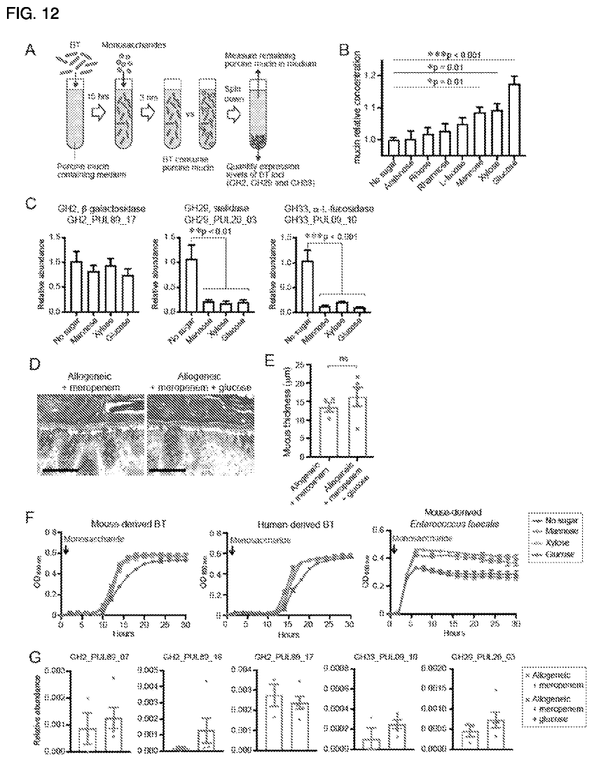

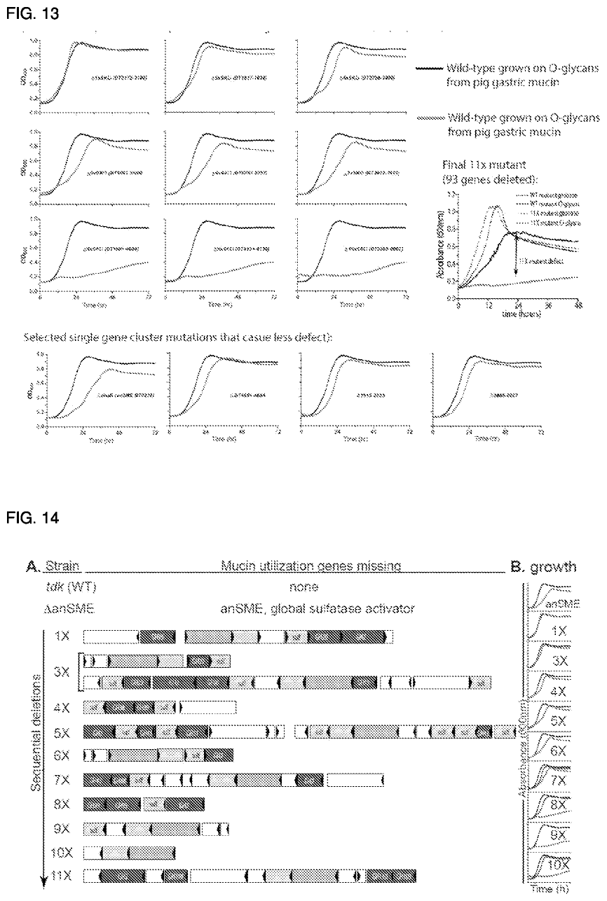

In the presence of multiple suitable carbohydrate substrates, BT has been found to preferentially consume certain carbohydrates first, and only after depleting these will it then upregulate utilization genes targeting other available polysaccharides (Rogers et al., 2013). Host mucin glycans are particularly low on the metabolic hierarchy and are typically targeted only after other dietary polysaccharides have been depleted (TerAvest et al., 2014). Both ambient carbohydrates as well as metabolic byproducts have been found to be modulators of the BT transcriptional profile (Schofield et al., 2018). It was determined if levels of soluble carbohydrates in the colonic lumen are perturbed by meropenem treatment, given that meropenem depletes the abundance of commensal Clostridia, which function to metabolize dietary fibers and starches (Chinda et al., 2004). Using ion chromatography-mass spectrometry (IC-MS), levels of luminal monosaccharides in the colon of mice were measured. It was found that meropenem treatment led to significantly lower concentrations of arabinose and xylose in allogeneic mice (FIG. 6B). To examine the effects of the presence of monosaccharides on mucin utilization by BT, BT was cultivated in bacterial media containing porcine gastric mucin, then subsequently a panel of monosaccharides was added levels of remaining mucin were quantified using a colorimetric assay (FIG. 12A). In the absence of additional monosaccharides, BT readily metabolized porcine gastric mucin. Mucin utilization by BT in the presence of certain monosaccharides, however, was significantly suppressed, including in particular mannose, glucose or xylose (FIG. 12B). Real-time PCR expression levels of GH2 β-galactosidase, GH33 sialidase and GH29 α-L-fucosidase were quantified; expression of GH33 sialidase and GH29 α-L-fucosidase by murine BT were suppressed by these monosaccharides, but expression of GH2 β-galactosidase was not significantly impacted (FIG. 12C). Altogether, these data indicate that following meropenem treatment, reductions in colonic monosaccharides, particularly xylose, may lead to increased mucus-degrading behavior by BT in the colon of allogeneic mice.

This led to a hypothesis that oral supplementation with xylose would mediate a benefit in allogeneic mice with GvHD aggravated by meropenem. Allogeneic mice were treated with meropenem as in FIG. 2F and with xylose from days 13 to 28 (FIG. 6C). Xylose supplementation prevented thinning of the colonic mucus thickness compared to allogeneic mice receiving meropenem alone, while a different monosaccharide, glucose, did not alter colonic mucus thickness (FIG. 6D and 6E and 12D and 12E), demonstrating that xylose could prevent thinning of the colonic mucus more than glucose. The relative abundances of BT were not significantly different in meropenem-treated mice with or without oral xylose supplementation (FIG. 6F). In addition, in vitro, growth of mouse-and human-derived BT in carbohydrate-poor media was augmented by xylose (FIG. 12F), indicating that xylose supplementation did not prevent thinning of the colonic mucus thickness via suppressing expansion of BT.

To profile effects of xylose on intestinal BT in the setting of GvHD, RNA sequencing of stool samples from meropenem-treated allogeneic mice without and with xylose supplementation was used to determine the mean relative abundances of expression of individual genes, comparing meropenem-treated allogeneic mice with and without xylose, and evaluated for a potential correlation with the mean relative abundances observed comparing allogeneic mice with and without meropenem, from FIG. 6A. A small amount of the changes in gene expression produced by meropenem could be attributed to changes in xylose (R=0.08), but the slope of the linear regression was significant (p=0.02, FIG. 6G). An examination of differentially abundant genes that could contribute to mucin glycan degradation revealed that one ORF, GH2_PUL89_16, was significantly downregulated by xylose supplementation in meropenem-treated allogeneic mice. Interestingly, meropenem-treated mice receiving glucose supplementation as a control monosaccharide did not show a similar effect (FIG. 6H and 12G). This ORF was not significantly upregulated by meropenem in allogeneic mice, however, and other enzymes that may contribute to mucin glycan degradation that were significantly upregulated by meropenem were not significantly downregulated by xylose. Overall, xylose supplementation does not substantially restore the spectrum of effects on BT seen with meropenem treatment. It was determined if oral supplementation with xylose could impact on GvHD severity in meropenem-treated allogeneic mice. Xylose supplementation significantly improved the survival of these mice (FIG. 6I). Together, these results indicate that xylose supplementation can inhibit expression of enzymes by BT that may contribute to mucus degradation, leading to better preservation of the mucus barrier and improved survival. This strategy provides an approach to ameliorate GvHD in the setting of an injured commensal microbiota following antibiotic treatment.

TABLE 1
Patient characteristics of allo-HSCT patients
who examined incidence of intestinal GvHD.

| | None | | Cefepime | | Meropenem | | Both | | |
|---|---|---|---|---|---|---|---|---|---|
| | (n = 80) | | (n = 124) | | (n = 32) | | (n = 59) | | P value |
| Median age (range), y | 57 | (20-72) | 55 | (18-77) | 57 | (25-72) | 54 | (19-74) | 0.8355 |
| Male, n (%) | 45 | (56%) | 69 | (57%) | 18 | (56%) | 40 | (68%) | 0.4332 |
| Donor type, n (%) | | | | | | | | | 0.0741 |
| MRD | 44 | (55%) | 59 | (48%) | 14 | (44%) | 14 | (24%) | |
| MUD | 33 | (41%) | 62 | (50%) | 18 | (56%) | 43 | (73%) | |
| MMRD | 2 | (3%) | 0 | (0%) | 0 | (0%) | 0 | (0%) | |
| MMUD | 1 | (1%) | 3 | (2%) | 0 | (0%) | 2 | (3%) | |
| Cell source | | | | | | | | | 0.2637 |
| Bone marrow | 21 | (26%) | 35 | (28%) | 5 | (16%) | 22 | (37%) | |
| Peripheral blood | 59 | (74%) | 89 | (72%) | 27 | (84%) | 37 | (63%) | |
| Conditioning, n (%) | | | | | | | | | 0.6650 |
| Myeloablative | 78 | (98%) | 118 | (95%) | 31 | (97%) | 54 | (92%) | |
| Non-myeloablative | 2 | (3%) | 6 | (5%) | 1 | (3%) | 5 | (9%) | |
| Acute GVHD | | | | | | | | | 0.9746 |
| Grade 0 | 33 | (41%) | 57 | (46%) | 13 | (41%) | 22 | (37%) | |
| Grade I | 18 | (23%) | 26 | (21%) | 5 | (16%) | 10 | (17%) | |
| Grade II | 23 | (29%) | 34 | (27%) | 10 | (31%) | 21 | (36%) | |
| Grade III-IV | 6 | (8%) | 7 | (6%) | 4 | (13%) | 6 | (10%) | |

Continuous variables are presented as the median and range, while categorical variables are presented as number and percentages. Non-repeated ANOVA was used to compare continuous variables, while chi-square or Fisher's exact tests were used to analyze the frequency distribution between categorical variables. P-values under 0.05 were considered to be statistically significant. MRD, HLA-matched related donor; MUD, HLA-matched unrelated donor; MMRD, HLA-mismatched related donor; MMUD, HLA-mismatched unrelated donor.

TABLE 2
Patient characteristics of allo-HSCT patients
who underwent intestinal microbiome profiling.

| | | |
|---|---|---|
| Meropenem- | Meropenem- | |
| untreated | treated | |
| (n = 26) | (n = 18) | P value |

| | | | | | |
|---|---|---|---|---|---|
| Median age (range), y | 53 | (39-72) | 58 | (38-70) | 0.0573 |
| Male, n (%) | 14 | (53%) | 8 | (44%) | 0.7591 |
| Disease, n (%) | | | | | 0.8961 |
| AML | 11 | (42%) | 9 | (50%) | |
| MDS | 4 | (15%) | 4 | (22%) | |
| MPD | 6 | (23%) | 3 | (16%) | |
| CML | 4 | (15%) | 0 | (0%) | |
| ALL | 0 | (0%) | 1 | (5%) | |
| Others | 1 | (3%) | 1 | (5%) | |
| Donor type, n (%) | | | | | 0.7327 |
| MRD | 8 | (30%) | 4 | (22%) | |
| MUD | 18 | (69%) | 14 | (77%) | |
| Cell source | | | | | 0.4390 |
| Bone marrow | 6 | (23%) | 2 | (11%) | |
| Peripheral blood | 20 | (76%) | 16 | (88%) | |
| Conditioning, n (%) | | | | | 1.0000 |
| Myeloablative | 26 | (100%) | 18 | (100%) | |
| Non-myeloablative | 0 | (0%) | 0 | (0%) | |
| Acute GVHD | | | | | 0.5531 |
| Grade 0 | 9 | (34%) | 7 | (38%) | |
| Grade I | 4 | (15%) | 0 | (0%) | |
| Grade II | 11 | (42%) | 7 | (38%) | |
| Grade III-IV | 2 | (7%) | 4 | (22%) | |

Continuous variables are presented as the median and range, while categorical variables are presented as number and percentages. Non-repeated ANOVA was used to compare continuous variables, while chi-square or Fisher's exact tests were used to analyze the frequency distribution between categorical variables. P-values under 0.05 were considered to be statistically significant. ALL, acute lymphoid leukemia; AML, acute myeloid leukemia; CML, chronic myeloid leukemia; MPD, myeloproliferative disorder; MRD, HLA-matched related donor; MDS, myelodysplastic syndrome; MUD, HLA-matched unrelated donor; MMRD, HLA-mismatched related donor; MMUD, HLA-mismatched unrelated donor.

TABLE 3
Quantification of the MIC of bacteria against meropenem.

| | MIC (μg/mL) |
|---|---|
| Bacteroides thetaiotaomicron (MDA-JAX BT001) | 4 |
| Bacteroides thetaiotaomicron (ATCC 29148) | 6 |
| Enterococcus faecalis (MDA-JAX EF001) | 12 |
| Clostridium disporicum (MDA-JAX CD001) | 0.094 |
| Clostridium saudiense (MDA-JAX CS001) | 0.38 |
| Lachnospiraceae unclassified (MDA-JAX LS001) | 0.38 |

TABLE 4
Formula per liter of BYEM10 broth.

| | | |
|---|---|---|
| KH₂PO₄ (P5655, Sigma-Aldrich) | 0.06 | g |
| K₂HPO₄ (RES20765, Sigma-Aldrich) | 0.06 | g |
| (NH₄)₂SO₄ (221260, Sigma-Aldrich) | 0.06 | g |
| NaCl (S3014, Sigma-Aldrich) | 0.12 | g |
| MgSO₄ (230391, Sigma-Aldrich) | 0.025 | g |
| CaCl₂ (746495, Sigma-Aldrich) | 0.01 | g |
| Yeast Extract (DF0127-17-9, Fisher Scientific) | 4.0 | g |
| Peptone from casein, pancreatic digest (70169, | 0.5 | g |
| Sigma-Aldrich) | | |
| Meat extract (70164, Sigma-Aldrich) | 0.375 | g |
| Brain Heart Infusion broth without dextrose (71-C00079, | 26 | g |
| Microbiology International) | | |
| Hemin (51280, Sigma-Aldrich) | 0.01 | g |
| L-Cysteine (30120, Sigma-Aldrich) | 1 | g |
| Vitamin Supplement (MD-VS, ATCC) | 10 | mL |
| Mineral Supplement (MDTMS, ATCC) | 10 | mL |
| Vitamin K3 (M5625, Sigma) | 1 | mg |

TABLE 5
Primers list for a BT mucin O-glycan deficient mutant.

| Primer | Sequence |
|---|---|
| BT0865 Forward | 5′-gcg gtc gac ggcaacctacctgtcttgtcg |
| BTO865 Reverse | 5′-tgacacatgagcattcgatagg |
| BT0867 Forward | 5′-cctatcgaatgctcatgtgtca cgattcgcgcggttgtgtgc |
| BTO867 Reverse | 5′-gcg tct aga cttatcacaccaaccaaaacagc |
| BT0752 Forward | 5′-gcg gtg gac gcgctgatggtggctgtcgta |
| BT0752 Reverse | 5′-aattccggttattaaaaccggaac |
| BT0757 Forward | 5′-gttccggttttaataaccggaatt gcataatgcaatagaaagcattac |
| BT0757 Reverse | 5′-gcg tct aga tcaggtttgttcaactcatccg |
| BT1617 Forward | 5′-gcg gtc gac ggaactggggtatcggtatgt |
| BT1617 Reverse | 5′-agtaaattttgtatcgaaccccg |
| BT1636 Forward | 5′-cggggttcgatacaaaatttact cgttacactaccggagaagaaa |
| BT1636 Reverse | 5′-gct tct aga agtgctttgaggttttcgttgc |
| BT2912 Forward | 5′-gcg gtc gac gggcattactattgtggtatgc |
| BT2912 Reverse | 5′-cccgctttactccattgatcc |
| BT2923 Forward | 5′-ggatcaatggagtaaagcggg ccgtatcattgggatacgttttg |
| BT2923 Reverse | 5′-gcg tct aga agtacgcgggtcaccaatagc |
| BT4681 Forward | 5′-gcg gtc gac gccccgtttggacaaagtagt |
| BT4681 Reverse | 5′-gttccttttgagatagttgacagg |
| BT4689 Forward | 5′-cctgtcaactatctcaaaaggaac acgatattgacttggaccatcc |
| BT4689 Reverse | 5′-gcg tct aga atacttccggttatttcgtgcc |
| BT3796 Forward | 5′-gcg gtc gac ggaaacattctcctggatggc |
| BT3796 Reverse | 5′-aattattcacctgtttagtcggg |
| BT3800 Forward | 5′-cccgactaaacaggtgaataatt cggagcggatgattatatgatg |
| BT3800 Reverse | 5′-gcg tct aga ttactcaattatcactggccgg |
| BT3172 Forward | 5′-gcg gtc gac cctccccaactgttgatgacc |
| BT3172 Reverse | 5′-agcgaagttagagtaaatcgacc |
| BT3180 Forward | 5′-ggtcgatttactctaacttcgct ggagcggtaatctctcctctc |
| BT3180 Reverse | 5′-gcg tct aga gattcttcttttcctgatgccg |
| BT3092 Forward | 5′-gcg gtc gac ccggtagaagtcggtcagcat |
| BT3092 Reverse | 5′-acgtttttattcccaagacttgc |
| BT3109 Forward | 5′-gcaagtcttgggaataaaaacgt ggaaactaaggaaaggagccc |
| BT3109 Reverse | 5′-gcg tct aga gtccggattcgtgtcttctcc |
| BT4636 Forward | 5′-GCG GTCGAC ctgccgacgtacaaatccgc |
| BT4636 Reverse | 5′-AGA ATT ATT ACT TAA ATT TGC CTG TC |
| BT4631 Forward | 5′-GACAGGCAAATTTAAGTAATAATTCTcatttattgaaacaacaaaatatatggc |
| BT4631 Reverse | 5′-GCG TCTAGA gtcccatcctgataatagtaataagg |
| BT4240 Forward | 5′-GCG TCTAGA ATCCTCGTGTTAATCGGAATG |
| BT4240 Reverse | 5′-TGTAACACAATTGAAAAGAGAGGCTCAAACGTATTCATACCC |
| BT4250 Forward | 5′-CTCTTTTCAATTGTGTTACA |
| BT4250 Reverse | 5′-GCG TCTAGA GGAACTACCTGATTCCATCCA |

### References

Altschul, S. F., Gish, W., Miller, W., Myers, E. W., and Lipman, D. J. (1990). Basic local alignment search tool. J Mol Biol 215, 403-410. 10.1016/S0022-2836(05)80360-2.

Arpaia, N., Campbell, C., Fan, X., Dikiy, S., van der Veeken, J., deRoos, P., Liu, H., Cross, J. R., Pfeffer, K., Coffer, P. J., and Rudensky, A. Y. (2013). Metabolites produced by commensal bacteria promote peripheral regulatory T-cell generation. Nature 504, 451-455. 10.1038/nature12726.

Atarashi, K., Tanoue, T., Shima, T., Imaoka, A., Kuwahara, T., Momose, Y., Cheng, G., Yamasaki, S., Saito, T., Ohba, Y., et al. (2011). Induction of colonic regulatory T cells by indigenous Clostridium species. Science 331, 337-341. 10.1126/science.1198469.

Bekker, V., Zwittink, R. D., Knetsch, C. W., Sanders, I., Berghuis, D., Heidt, P. J., Vossen, J., de Vos, W. M., Belzer, C., Bredius, R. G. M., et al. (2019). Dynamics of the Gut Microbiota in Children Receiving Selective or Total Gut Decontamination Treatment during Hematopoietic Stem Cell Transplantation. Biol Blood Marrow Transplant 25, 1164-1171. 10.1016/j.bbmt.2019.01.037.

Benjdia, A., Martens, E. C., Gordon, J. I., and Berteau, O. (2011). Sulfatases and a radical S35 adenosyl-L-methionine (AdoMet) enzyme are key for mucosal foraging and fitness of the prominent human gut symbiont, Bacteroides thetaiotaomicron. J Biol Chem 286, 25973-25982. 10.1074/jbc.M111.228841.

Bergstrom, K. S., and Xia, L. (2013). Mucin-type O-glycans and their roles in intestinal homeostasis. Glycobiology 23, 1026-1037. 10.1093/glycob/cwt045.

Bjursell, M. K., Martens, E. C., and Gordon, J. I. (2006). Functional genomic and metabolic studies of the adaptations of a prominent adult human gut symbiont, Bacteroides thetaiotaomicron, to the suckling period. J Biol Chem 281, 36269-36279. 10.1074/jbc.M606509200.

Bloom, S. M., Bijanki, V. N., Nava, G. M., Sun, L., Malvin, N. P., Donermeyer, D. L., Dunne, W. M., Jr., Allen, P. M., and Stappenbeck, T. S. (2011). Commensal Bacteroides species induce colitis in host-genotype-specific fashion in a mouse model of inflammatory bowel disease. Cell Host Microbe 9, 390-403. 10.1016/j.chom.2011.04.009.

Buchfink, B., Xie, C., and Huson, D. H. (2015). Fast and sensitive protein alignment using DIAMOND. Nat Methods 12, 59-60. 10.1038/nmeth.3176.

Caporaso, J. G., Kuczynski, J., Stombaugh, J., Bittinger, 5 K., Bushman, F. D., Costello, E. K., Fierer, N., Pena, A. G., Goodrich, J. K., Gordon, J. I., et al. (2010). QIIME allows analysis of high throughput community sequencing data. Nat Methods 7, 335-336. 10.1038/nmeth.f.303.

Caporaso, J. G., Lauber, C. L., Walters, W. A., Berg-Lyons, D., Huntley, J., Fierer, N., Owens, S. M., Betley, J., Fraser, L., Bauer, M., et al. (2012). Ultra-high-throughput microbial community analysis on the Illumina HiSeq and MiSeq platforms. ISME J 6, 1621-1624. 10.1038/ismej.2012.8.

Carver, T., Thomson, N., Bleasby, A., Berriman, M., and Parkhill, J. (2009). DNAPlotter: circular and linear interactive genome visualization. Bioinformatics 25, 119-120. 10.1093/bioinformatics/btn578.

Chinda, D., Nakaji, S., Fukuda, S., Sakamoto, J., Shimoyama, T., Nakamura, T., Fujisawa, T., 15 Terada, A., and Sugawara, K. (2004). The fermentation of different dietary fibers is associated with fecal clostridia levels in men. J Nutr 134, 1881-1886. 10.1093/jn/134.8.1881.

Cooke, K. R., Kobzik, L., Martin, T. R., Brewer, J., Delmonte, J., Jr., Crawford, J. M., and Ferrara, J. L. (1996). An experimental model of idiopathic pneumonia syndrome after bone marrow transplantation: I. The roles of minor H antigens and endotoxin. Blood 88, 3230-3239.

Dodd, D., Mackie, R. I., and Cann, I. K. (2011). Xylan degradation, a metabolic property shared by rumen and human colonic Bacteroidetes. Mol Microbiol 79, 292-304. 10.1111/j.1365-2958.2010.07473.x.

Donaldson, G. P., Chou, W. C., Manson, A. L., Rogov, P., Abeel, T., Bochicchio, J., Ciulla, D., Melnikov, A., Ernst, P. B., Chu, H., et al. (2020). Spatially distinct physiology of Bacteroides fragilis within the proximal colon of gnotobiotic mice. Nat Microbiol 5, 746-756. 10.1038/s41564-020-0683-3.

Edgar, R. C. (2016). UNOISE2: improved error-correction for Illumina 16S and ITS amplicon sequencing. bioRxiv, 081257. 10.1101/081257.

Elgarten, C. W., Li, Y., Getz, K. D., Hemmer, M., Huang, Y. V., Hall, M., Wang, T., Kitko, C. L., Jagasia, M. H., Nishihori, T., et al. (2021). Broad spectrum antibiotics and risk of graft-versus-host disease in pediatric patients transplanted for acute leukemia: association of carbapenem use with risk of acute GvHD. Transplant Cell Ther 27, 177 e171-177 e178. 10.1016/j.jtct.2020.10.012.

Freifeld, A. G., Bow, E. J., Sepkowitz, K. A., Boeckh, M. J., Ito, J. I., Mullen, C. A., Raad, II, Rolston, K. V., Young, J. A., Wingard, J. R., and Infectious Diseases Society of, A. (2011). Clinical practice guideline for the use of antimicrobial agents in neutropenic patients with cancer: 2010 Update by the Infectious Diseases Society of America. Clin Infect Dis 52, 427-431. 10.1093/cid/ciq 147.

Hayase, E., Hashimoto, D., Nakamura, K., Noizat, C., Ogasawara, R., Takahashi, S., Ohigashi, H., Yokoi, Y., Sugimoto, R., Matsuoka, S., et al. (2017). R-Spondin1 expands Paneth cells and prevents dysbiosis induced by graft-versus-host disease. J Exp Med 214, 3507-3518. 40 10.1084/jem.20170418.

Hickey, C. A., Kuhn, K. A., Donermeyer, D. L., Porter, N. T., Jin, C., Cameron, E. A., Jung, H., Kaiko, G. E., Wegorzewska, M., Malvin, N. P., et al. (2015). Colitogenic Bacteroides thetaiotaomicron Antigens Access Host Immune Cells in a Sulfatase-Dependent Manner via Outer Membrane Vesicles. Cell Host Microbe 17, 672-680. 10.1016/j.chom.2015.04.002.

Hidaka, D., Hayase, E., Shiratori, S., Hasegawa, Y., Ishio, T., Tateno, T., Okada, K., Goto, H., Sugita, J., Onozawa, M., et al. (2018). The association between the incidence of intestinal graftvs-host disease and antibiotic use after allogeneic hematopoietic stem cell transplantation. Clin Transplant 32, e13361. 10.1111/ctr.13361.

Hill, G. R., and Ferrara, J. L. (2000). The primacy of the gastrointestinal tract as a target organ of acute graft-versus-host disease: rationale for the use of cytokine shields in allogeneic bone marrow transplantation. Blood 95, 2754-2759.

Holler, E., Butzhammer, P., Schmid, K., Hundsrucker, C., Koestler, J., Peter, K., Zhu, W., Sporrer, D., Hehlgans, T., Kreutz, M., et al. (2014). Metagenomic 5 analysis of the stool microbiome in patients receiving allogeneic stem cell transplantation: loss of diversity is associated with use of systemic antibiotics and more pronounced in gastrointestinal graft-versus-host disease. Biol Blood Marrow Transplant 20, 640-645. 10.1016/j.bbmt.2014.01.030.

Hooper, L. V., Littman, D. R., and Macpherson, A. J. (2012). Interactions between the microbiota and the immune system. Science 336, 1268-1273. 10.1126/science.1223490.

Jenq, R. R., Ubeda, C., Taur, Y., Menezes, C. C., Khanin, R., Dudakov, J. A., Liu, C., West, M. L., Singer, N. V., Equinda, M. J., et al. (2012). Regulation of intestinal inflammation by microbiota following allogeneic bone marrow transplantation. J Exp Med 209, 903-911. 10.1084/jem.20112408.

Jovel, J., Patterson, J., Wang, W., Hotte, N., O'Keefe, S., Mitchel, T., Perry, T., Kao, D., Mason, A. L., Madsen, K. L., and Wong, G. K. (2016). Characterization of the Gut Microbiome Using 16S or Shotgun Metagenomics. Front Microbiol 7, 459. 10.3389/fmicb.2016.00459.

Kilcoyne, M., Gerlach, J. Q., Farrell, M. P., Bhavanandan, V. P., and Joshi, L. (2011). Periodic acid-Schiff's reagent assay for carbohydrates in a microtiter plate format. Anal Biochem 416, 18-26. 10.1016/j.ab.2011.05.006.

Kolmogorov, M., Yuan, J., Lin, Y., and Pevzner, P. A. (2019). Assembly of long, error-prone reads using repeat graphs. Nat Biotechnol 37, 540-546. 10.1038/s41587-019-0072-8.

Koropatkin, N. M., Martens, E. C., Gordon, J. I., and Smith, T. J. (2008). Starch catabolismby a prominent human gut symbiont is directed by the recognition of amylose helices. Structure 16, 25 1105-1115. 10.1016/j.str.2008.03.017.

Koyama, M., Cheong, M., Markey, K. A., Gartlan, K. H., Kuns, R. D., Locke, K. R., Lineburg, K. E., Teal, B. E., Leveque-El Mouttie, L., Bunting, M. D., et al. (2015). Donor colonic CD103+ dendritic cells determine the severity of acute graft-versus-host disease. J Exp Med 212, 1303-1321. 10.1084/jem.20150329.

Lee, S. E., Lim, J. Y., Ryu, D. B., Kim, T. W., Park, S. S., Jeon, Y. W., Yoon, J. H., Cho, B. S., Eom, K. S., Kim, Y. J., et al. (2019). Alteration of the Intestinal Microbiota by Broad-Spectrum Antibiotic Use Correlates with the Occurrence of Intestinal Graft-versus-Host Disease. Biol Blood Marrow Transplant 25, 1933-1943. 10.1016/j.bbmt.2019.06.001.

Ley, R. E., Lozupone, C. A., Hamady, M., Knight, R., and Gordon, J. I. (2008). Worlds within worlds: evolution of the vertebrate gut microbiota. Nat Rev Microbiol 6, 776-788. 10.1038/nrmicro1978.

Li, H., and Durbin, R. (2009). Fast and accurate short read alignment with Burrows-Wheeler transform. Bioinformatics 25, 1754-1760. 10.1093/bioinformatics/btp324.

Lozupone, C., Lladser, M. E., Knights, D., Stombaugh, J., and Knight, R. (2011). UniFrac: an effective distance metric for microbial community comparison. ISME J 5, 169-172. 10.1038/ismej.2010.133.

Martens, E. C., Chiang, H. C., and Gordon, J. I. (2008). Mucosal glycan foraging enhances fitness and transmission of a saccharolytic human gut bacterial symbiont. Cell Host Microbe 4, 447-457. 10.1016/j.chom.2008.09.007.

Mathewson, N. D., Jenq, R., Mathew, A. V., Koenigsknecht, M., Hanash, A., Toubai, T., Oravecz-Wilson, K., Wu, S. R., Sun, Y., Rossi, C., et al. (2016). Gut microbiome-derived metabolites modulate intestinal epithelial cell damage and mitigate graft-versus-host disease. Nat Immunol 17, 505-513. 10.1038/ni.3400.

Nava, G. M., and Stappenbeck, T. S. (2011). Diversity of the autochthonous colonic microbiota. Gut Microbes 2, 99-104. 10.4161/gmic.2.2.15416.

Okumura, R., Kurakawa, T., Nakano, T., Kayama, H., Kinoshita, M., Motooka, D., Gotoh, K., Kimura, T., Kamiyama, N., Kusu, T., et al. (2016). Lypd8 promotes the segregation of flagellated microbiota a 5 nd colonic epithelia. Nature 532, 117-121. 10.1038/nature17406.

Peled, J. U., Gomes, A. L. C., Devlin, S. M., Littmann, E. R., Taur, Y., Sung, A. D., Weber, D., Hashimoto, D., Slingerland, A. E., Slingerland, J. B., et al. (2020). Microbiota as Predictor of Mortality in Allogeneic Hematopoietic-Cell Transplantation. N Engl J Med 382, 822-834. 10.1056/NEJMoa1900623.

Przepiorka, D., Weisdorf, D., Martin, P., Klingemann, H. G., Beatty, P., Hows, J., and Thomas, E. D. (1995). 1994 Consensus Conference on Acute GvHD Grading. Bone Marrow Transplant 15, 825-828.

Quast, C., Pruesse, E., Yilmaz, P., Gerken, J., Schweer, T., Yarza, P., Peplies, J., and Glockner, F. O. (2013). The SILVA ribosomal RNA gene database project: improved data processing and web-based tools. Nucleic Acids Res 41, D590-596. 10.1093/nar/gks1219.

Rogers, T. E., Pudlo, N. A., Koropatkin, N. M., Bell, J. S., Moya Balasch, M., Jasker, K., and Martens, E. C. (2013). Dynamic responses of Bacteroides thetaiotaomicron during growth on glycan mixtures. Mol Microbiol 88, 876-890. 10.1111/mmi.12228.

Rognes, T., Flouri, T., Nichols, B., Quince, C., and Mahe, F. (2016). VSEARCH: a versatile open source tool for metagenomics. PeerJ 4, e2584. 10.7717/peerj.2584.

Schloss, P. D., Westcott, S. L., Ryabin, T., Hall, J. R., Hartmann, M., Hollister, E. B., Lesniewski, R. A., Oakley, B. B., Parks, D. H., Robinson, C. J., et al. (2009). Introducing mothur: open-source, platform-independent, community-supported software for describing and comparing microbial communities. Appl Environ Microbiol 75, 7537-7541. 10.1128/AEM.01541-09.

Schofield, W. B., Zimmermann-Kogadeeva, M., Zimmermann, M., Barry, N. A., and Goodman, A. L. (2018). The Stringent Response Determines the Ability of a Commensal Bacterium to Survive Starvation and to Persist in the Gut. Cell Host Microbe 24, 120-132 e126. 10.1016/j.chom.2018.06.002.

Schroeder, T. H., Reiniger, N., Meluleni, G., Grout, M., Coleman, F. T., and Pier, G. B. (2001). Transgenic cystic fibrosis mice exhibit reduced early clearance of Pseudomonas aeruginosa from the respiratory tract. J Immunol 166, 7410-7418. 10.4049/jimmunol.166.12.7410.

Schwab, L., Goroncy, L., Palaniyandi, S., Gautam, S., Triantafyllopoulou, A., Mocsai, A., Reichardt, W., Karlsson, F. J., Radhakrishnan, S. V., Hanke, K., et al. (2014). Neutrophil granulocytes recruited upon translocation of intestinal bacteria enhance graft-versus-host disease via tissue damage. Nat Med 20, 648-654. 10.1038/nm.3517.

Shono, Y., Docampo, M. D., Peled, J. U., Perobelli, S. M., Velardi, E., Tsai, J. J., Slingerland, A. E., Smith, O. M., Young, L. F., Gupta, J., et al. (2016). Increased GvHD-related mortality with broad spectrum antibiotic use after allogeneic hematopoietic stem cell transplantation in human patients and mice. Sci Transl Med 8, 339ra371. 10.1126/scitranslmed.aaf2311.

Simms-Waldrip, T. R., Sunkersett, G., Coughlin, L. A., Savani, M. R., Arana, C., Kim, J., Kim, M., Zhan, X., Greenberg, D. E., Xie, Y., et al. (2017). Antibiotic-Induced Depletion of Anti inflammatory Clostridia Is Associated with the Development of Graft-versus-Host Disease in Pediatric Stem Cell Transplantation Patients. Biol Blood Marrow Transplant 23, 820-829. 10.1016/j.bbmt.2017.02.004.

Smith, P. M., Howitt, M. R., Panikov, N., Michaud, M., Gallini, C. A., Bohlooly, Y. M., Glickman, J. N., and Garrett, W. S. (2013). The microbial metabolites, short-chain fatty acids, regulate colonic Treg cell homeostasis. Science 341, 569-573. 10.1126/science.1241165.

Sonnenburg, J. L., Xu, J., Leip, D. D., Chen, C. H., Westover, B. P., Weatherford, J., Buhler, J. D., and Gordon, J. I. (2005). Glycan foraging in vivo by an intestine-adapted bacterial symbiont. Science 307, 1955-1959. 10.1126/science.1109051.

Stothard, P. (2000). The sequence manipulation suite: JavaScript programs for analyzing and formatting protein and D 5 NA sequences. Biotechniques 28, 1102, 1104. 10.2144/00286ir01.

Tailford, L. E., Crost, E. H., Kavanaugh, D., and Juge, N. (2015). Mucin glycan foraging in the human gut microbiome. Front Genet 6, 81. 10.3389/fgene.2015.00081.

Taplitz, R. A., Kennedy, E. B., Bow, E. J., Crews, J., Gleason, C., Hawley, D. K., Langston, A. A., Nastoupil, L. J., Rajotte, M., Rolston, K. V., et al. (2018). Antimicrobial Prophylaxis for Adult Patients With Cancer-Related Immunosuppression: ASCO and IDSA Clinical Practice Guideline Update. J Clin Oncol 36, 3043-3054. 10.1200/JCO.18.00374.

Tatusova, T., DiCuccio, M., Badretdin, A., Chetvernin, V., Nawrocki, E. P., Zaslavsky, L., Lomsadze, A., Pruitt, K. D., Borodovsky, M., and Ostell, J. (2016). NCBI prokaryotic genome annotation pipeline. Nucleic Acids Res 44, 6614-6624. 10.1093/nar/gkw569.

Taur, Y., Jenq, R. R., Perales, M. A., Littmann, E. R., Morjaria, S., Ling, L., No, D., Gobourne, A., Viale, A., Dahi, P. B., et al. (2014). The effects of intestinal tract bacterial diversity on mortality following allogeneic hematopoietic stem cell transplantation. Blood 124, 1174-1182. 10.1182/blood-2014-02-554725.

TerAvest, M. A., He, Z., Rosenbaum, M. A., Martens, E. C., Cotta, M. A., Gordon, J. I., and Angenent, L. T. (2014). Regulated expression of polysaccharide utilization and capsular biosynthesis loci in biofilm and planktonic Bacteroides thetaiotaomicron during growth in chemostats. Biotechnol Bioeng 111, 165-173. 10.1002/bit.24994.

Terrapon, N., Lombard, V., Drula, E., Lapebie, P., Al-Masaudi, S., Gilbert, H. J., and Henrissat, B. (2018). PULDB: the expanded database of Polysaccharide Utilization Loci. Nucleic Acids Res 46, D677-D683. 10.1093/nar/gkx1022.

van der Hee, B., and Wells, J. M. (2021). Microbial Regulation of Host Physiology by Short-chain Fatty Acids. Trends Microbiol 29, 700-712. 10.1016/j.tim.2021.02.001.

Wick, R. R., Judd, L. M., and Holt, K. E. (2019). Performance of neural network basecalling tools for Oxford Nanopore sequencing. Genome Biol 20, 129. 10.1186/s13059-019-1727-y.

Wrzosek, L., Miquel, S., Noordine, M. L., Bouet, S., Joncquel Chevalier-Curt, M., Robert, V., Philippe, C., Bridonneau, C., Cherbuy, C., Robbe-Masselot, C., et al. (2013). Bacteroides thetaiotaomicron and Faecalibacterium prausnitzii influence the production of mucus glycans and the development of goblet cells in the colonic epithelium of a gnotobiotic model rodent. BMC Biol 11, 61. 10.1186/1741-7007-11-61.

Yang, Y. W., Chen, M. K., Yang, B. Y., Huang, X. J., Zhang, X. R., He, L. Q., Zhang, J., and Hua, Z. C. (2015). Use of 16S rRNA Gene-Targeted Group-Specific Primers for Real-Time PCR Analysis of Predominant Bacteria in Mouse Feces. Appl Environ Microbiol 81, 6749-6756. 10.1128/AEM.01906-15.

All publications and patents mentioned in the present application are herein incorporated by reference. Various modification and variation of the described methods and compositions of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled in the relevant fields are intended to be within the scope of the following claims.

1. A composition, comprising:
a non-mucin degrading Bacteroides thetaiotaomicron comprising a mutation that deletes at least one polysaccharide utilization loci (PUL) selected from the group consisting of BT3172-80, BT1617-36, BT3796-3800, BT3092-3109, BT0752-57, BT2912-23, BT4681-84, BT4634-31, BT0865-67, and BT4250-40.

2. The composition of claim 1, wherein PULs BT3172-80, BT1617-36, BT3796-3800, BT3092-3109, BT0752-57, BT2912-23, and BT4681-84 are deleted.

3. The composition of claim 1, wherein PULs of BT3172-80, BT1617-36, BT3796-3800, BT3092-3109, BT0752-57, BT2912-23, BT4681-84, and BT4634-31 are deleted.

4. The composition of claim 1, wherein PULs BT3172-80, BT1617-36, BT3796-3800, BT3092-3109, BT0752-57, BT2912-23, BT4681-84, BT4634-31, and BT0865-67 are deleted.

5. The composition of claim 1, wherein PULs BT3172-80, BT1617-36, BT3796-3800, BT3092-3109, BT0752-57, BT2912-23, BT4681-84, BT4634-31, BT0865-67, and BT4250-40 are deleted.

6. The composition of claim 1, wherein said PULs encode one or more enzymes selected from the group consisting of GH95, sulfatase, GH20, GH2, GH18, GH36, GH29, GH51, GH2, GH88, GH95, GH109, and GH110.

7. The composition of claim 1, wherein said composition is a pharmaceutical composition.

8. The composition of claim 4, wherein said composition further comprises a pharmaceutically acceptable carrier.

9. The composition of claim 1, wherein said composition is a supplement or nutraceutical.

10. The composition of claim 1, wherein said composition further comprises one or more polysaccharides selected from the group consisting of arabinan, arabinogalactan, homogalacturonan, rhamnogalacturonan I, pectic galactan, chondroitin sulfate, dextran, α-mannan, and levan.

11. A composition, comprising:
a non-mucin degrading bacteria selected from the group consisting of Bacteroides caccae Bacteroides fragilis, Bacteroides vulgatus, Bacteroides dorei, Bacteroides fluxus, Bacteroides massiliensis, Bacteroides nordii, Bacteroides ovatus, Parabacteroides merdae, Parabacteroides distasonis, Parabacteroides goldsteinii, and Parabacteroides gordonii
Wherein said bacteria comprises a mutation that inactivates or eliminates expression of at least one polysaccharide utilization loci (PUL) related to degradation of mucin.

12-13. (canceled)

14. A method of treating or preventing an inflammatory disease, comprising:
administering the composition of claim 1 to a subject in need thereof.

15. The method of claim 14, wherein said inflammatory disorder is selected from the group consisting of graft vs host disease, inflammatory bowel disease, and Crohn's disease.

16. The method of claim 14, wherein said subject has undergone an organ transplant.

17. The method of claim 16, wherein said composition is administered prior to said organ transplant, after said organ transplant, or both.

18. The method of claim 14, wherein said composition is administered in multiple doses.

19. The method of claim 14, further comprising administering an additional treatment for said inflammatory disease.

20. The method of claim 19, wherein said additional treatment is a corticosteroid.

21-22. (canceled)

23. The method of claim 14, wherein said composition is administered orally or fecally.

24. The method of claim 14, wherein said composition is in a capsule.

25-26. (canceled)


SEQUENCE LISTING (8 sequences)

SEQ ID NO: 1 - DNA, 20 bp, synthetic construct
  source 1..20: mol_type = other DNA
cgcactcttcttgcatctgc

SEQ ID NO: 2 - DNA, 20 bp, synthetic construct
  source 1..20: mol_type = other DNA
taccaacggctcacattggg

SEQ ID NO: 3 - DNA, 20 bp, synthetic construct
  source 1..20: mol_type = other DNA
gatgctggaaaaggcaacgg

SEQ ID NO: 4 - DNA, 20 bp, synthetic construct
  source 1..20: mol_type = other DNA
agcgtgccttttccttctga

SEQ ID NO: 5 - DNA, 25 bp, synthetic construct
  source 1..25: mol_type = other DNA
ggtcaccgaaagacattattcatcg

SEQ ID NO: 6 - DNA, 23 bp, synthetic construct
  source 1..23: mol_type = other DNA
gccgtttgatacagatccattcc

SEQ ID NO: 7 - DNA, 21 bp, synthetic construct
  source 1..21: mol_type = other DNA
cacaacagccatagcgttcca

SEQ ID NO: 8 - DNA, 23 bp, synthetic construct
  source 1..23: mol_type = other DNA
atcgcaaaaataagatgggcaaa